SEQUENCE LISTING (1 sequence)

SEQ ID NO: 1 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

(19) United States
(12) Patent Application Publication
(10) Pub. No.: US 2024/0151727 A1
(43) Pub. Date: May 9, 2024

(54) DIAGNOSTIC ALGORITHMS FOR DETERMINING AND TREATING CLINICALLY SIGNIFICANT PROSTATE CANCER BASED ON SIALYLATED AND FUCOSYLATED PROSTATE SPECIFIC ANTIGEN

(71) Applicant: HIGH DEVELOPMENT LIMITED, Kowloon Bay (HK)

(72) Inventors: John Klassen, Edmonton (CA); Zhixiong Li, Edmonton (CA)

(73) Assignee: HIGH DEVELOPMENT LIMITED, Kowloon Bay (HK)

(21) Appl. No.: 18/098,435

(22) Filed: Jan. 18, 2023

Related U.S. Application Data
(60) Provisional application No. 63/423,766, filed on Nov. 8, 2022.

(51) Int. Cl. G01N 33/574; G01N 33/573; G01N 33/68

(52) U.S. Cl. CPC G01N 33/57488; G01N 33/573; G01N 33/57434; G01N 33/6851; C12Y 302/01018; G01N 2333/936

(57) ABSTRACT

Provided herein are an α2-3 disialylated PSA protein standard and an α2-6 disialylated PSA protein standard, as well as various methods for measuring the relative (fractional) amount of the total N-acetylneuraminic acid (Neu5Ac) that is α2-3-linked N-acetylneuraminic acid (α2-3-linked Neu5Ac) and is α2-6-linked N-acetylneuraminic acid (α2-6-linked Neu5Ac) on PSA present in a blood serum or plasma sample, which is useful for determining whether a subject has a low risk (GG1), intermediate risk (GG2) or a high risk (GG3, 4 or 5) prostate cancer and/or determining and/or treating whether a human subject suspected of having prostate cancer should receive a needle biopsy. Also included are methods of monitoring and treating a prostate cancer patient post prostate cancer removal surgery for need of additional cancer treatment.

This is a Track I Prioritized Examination—Nonprovisional Application which claims benefit to Provisional Application No. 63/423,766 filed Nov. 8, 2022 (pending), which is incorporated herein in their entirety.

### RELATED APPLICATIONS

This application claims priority to U.S. provisional application 63/423,766, which was filed on Nov. 8, 2022, the contents of which are incorporated herein in their entirety.

### BACKGROUND OF THE INVENTION

Prostate cancer (PCa) is the second most commonly diagnosed cancer in men and the fifth leading cause of cancer deaths in men.^{1,2} The serum prostate-specific antigen (PSA) test is the most commonly used test for PCa screening and has greatly reduced PCa-related mortality.^{3,4} The prostate specific antigen (PSA) test, which measures PSA levels in blood, is ubiquitously used for PCa screening. However, because PSA levels may be elevated for reasons other than PCa, it leads to high rates of misdiagnosis and overtreatment. Moreover, the PSA test is not specific for clinically significant forms of PCa, histologically classified as Gleason Group 2 (GG2/intermediate-risk PCa) and Gleason Groups 3, 4, and 5 (GG3/4/5/high-risk PCa). The serum PSA test has a false positive rate of 35-45% when using a cut-off of ≥4 ng/mL, causing many individuals to undergo a needle biopsy procedure that often yields no evidence of clinically significant PCa.⁵⁻⁷ More men will die from the side effects of a prostate needle biopsy (0.08% mortality) than from low-risk PCa (Gleason Group 1/GG1) (<0.01% mortality), making it the most lethal biopsy procedure in medicine.⁸⁻¹¹ There is an urgent need for more accurate biomarkers that distinguish patients with clinically significant PCa from all other diseases of the prostate, such as low-risk PCa (GG1), prostatitis, and benign prostatic hyperplasia (BPH).

Aberrant protein glycosylation is a feature common to all cancers and altered PSA glycosylation has been observed in PCa.^{7,12,13} PSA has a single N-glycosylation site (Asn-69), which is occupied primarily by fucosylated or non-fucosylated complex-type disialylated bi-antennary N-glycans, comprised of α2-3- or α2-6-linked sialic acid (N-acetylneuraminic acid, Neu5Ac)^{7,14,15} There is growing evidence of altered N-glycans on PSA secreted by PCa cells, which are different from the N-glycans on PSA secreted from normal prostatic epithelium.¹⁶ These differences include increased levels of α2-3-linked Neu5Ac,¹⁷⁻²¹ and increases in hybrid, oligomannose, and bi-antennary digalactosylated monosialylated glycans, bisecting and mono-antennary glycans, and multisialylated LacdiNAc,^{16-18,21-23} and these levels appear to increase in a graded fashion alongside PCa progression.^{22,23} Indeed, altered PSA glycosylation is a promising biomarker for distinguishing clinically significant PCa from all other prostatic diseases.^{16,24-27} For example, recent studies on the relative content of α2-3-linked Neu5Ac and core fucosylation of serum PSA, independently or together, is specific for GG4 PCa.^{13,24-26,28,29} However, it is not clear if α2-3-linked Neu5Ac and core fucosylation is specific for clinically significant PCa, histologically classified as Gleason Group 2-5 (GG2-5) disease. Far more patients are diagnosed with GG2 and GG3 disease than GG4 disease, risk types that are often confused for low-risk PCa (GG1) by serum PSA and other blood tests (PHI, 4K Score).^{30,31}

Typically, PSA glycosylation analysis is performed on released N-glycans or glycopeptides using high performance liquid chromatography (HPLC) or capillary electrophoresis (CE) separation, combined with mass spectrometry (MS) detection. 32-34 For example, PSA tryptic glycopeptides can be separated using hydrophilic interaction liquid chromatography (HILIC) or by CE.^{32,34,35} Separation of isomeric glycopeptides containing α2-3- and α2-6-linked Neu5Ac, and their relative quantification by HILIC, coupled with multiple reaction monitoring MS, has been demonstrated with identification of the Neu5Ac linkages achieved through non-specific and α2-3-linked Neu5Ac-specific neuraminidases.³²

While commonly used for PSA glycosylation analysis, the bottom-up LC/CE-MS workflow involves multiple steps, including enzymatic digestion and N-glycan/glycopeptide enrichment.³⁶ Such bottom-up strategies are labor intensive to implement, suffer from sample loss(es) and, most importantly, are susceptible to contamination by other glycoproteins that co-purify with PSA.^{36,37} In contrast, top-down approaches, based on the glycan analysis of intact PSA, avoid these sources of error. 15 For example, lectin-based immunoassays have been used to quantify the α2-3-linked Neu5Ac content of intact PSA for the diagnosis of PCa.³² Currently, the most sensitive diagnostic tests include detection of α2-3-linked sialic acid in intact PSA using anti-α2-3-linked Neu5Ac antibody or Maackia amurensis (MAA)-agarose lectin with luminescence as the readout.^{24,26} However, lectin-based assays underestimate α2-3-linked Neu5Ac content owing to the inability to accurately quantify bi-antennary N-glycans possessing both α2-3- and α2-6-linked Neu5Ac. Recently, LC-MS analysis of intact PSA for the determination of glycan composition was demonstrated. 15 This approach does not allow for the α2-3- and α2-6-linked Neu5Ac content of PSA to be quantified, thus is not sufficiently quantitative for clinical practice. This is essential because it is the ratio of the two Neu5Ac linkage states that is hypothesized to discriminate between GG1 disease (low-risk) from GG2-5 disease (intermediate- to high-risk PCa), also known as clinically significant PCa.^{25-31,38}

### SUMMARY OF THE INVENTION

Provided herein are an α2-3 disialylated PSA protein standard (23PSA) having the amino acid sequence set forth in SEQ ID NO: 1 (see FIG. 5) and an α2-6 disialylated PSA protein standard (26PSA) having the amino acid sequence set forth in SEQ ID NO: 1 (see FIG. 5).

The invention provides various methods for measuring the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample. This measurement then provides the necessary information to determine whether a subject should receive a needle biopsy, other diagnostic tests and/or further cancer treatments. The measuring and quantifying are performed with electrospray ionization mass spectrometry (ESI-MS) and center-of-mass (CoM) analysis of the PSA and quantification of the fractional amount of α2-3-linked Neu5Ac in PSA from the CoM analysis is used in the methods of the invention.

The inventions include quantifying the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample obtained from a human subject suspected of having prostate cancer to characterize whether the subject has a low risk (GG1), intermediate risk (GG2) or a high risk (GG3, 4 or 5) prostate cancer. Using the fractional amount of total Neu5Ac on PSA that is α2-3-linked Neu5Ac one can predict the subject as having clinically significant prostate cancer (Gleason Group 2, 3, 4 and 5 also known as GG2-5). When the fraction (percentage) of Neu5Ac on PSA that is α2-3-linked Neu5Ac is less than 0.240 (24.0%), then the subject has a high likelihood (>95% chance) of low risk (GG1) prostate cancer or no prostate cancer. When the fraction (percentage) is equal to or above 0.240 (24.0%) and less than or equal to 0.280 (28.0%), then the subject has a low likelihood of clinically significant prostate cancer (GG2-5); and when the fraction (percentage) is above 0.280 (28.0%), then the subject has a high likelihood (˜90%) of clinically significant prostate cancer (GG2-5).

Also included is a method of determining whether a human subject suspected of having prostate cancer should receive a needle biopsy. This involves quantification of the fractional amount of α2-3-linked Neu5Ac in PSA from the CoM analysis; and when the fraction (percentage) of total Neu5Ac on PSA that is α2-3-linked Neu5Ac is greater than 0.280 (28.0%) recommend the subject proceed immediately to a needle biopsy and forego the need for a diagnostic Mill scan; and when the fraction (percentage) is less than 0.240 (24.0%) recommend no diagnostic MRI scan and no needle biopsy.

A further step can be employed. When the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated; and if the fraction (percentage) of fucosylated PSA is greater than 0.640 (64.0%), then recommend the subject have a needle biopsy and recommend to forego an mpMRI scan; and if the fraction (percentage) of fucosylated PSA is less than or equal to 0.640 (64.0%), then recommend the subject have an mpMRI; and when the mpMRI shows a PI-RADS score of 3, 4 or 5, then recommend a needle biopsy and if the PI-RADS score is 1 or 2 then recommend no needle biopsy.

A method of treating a human subject with a diagnostic treatment is also provided. When the fraction (percentage) of total Neu5Ac on PSA that is α2-3-linked Neu5Ac is greater than 0.280 (28.0%) treat the subject with a needle biopsy and forego a diagnostic MRI scan. When the fraction (percentage) is less than 0.240 (24.0%) do not treat subject with a needle biopsy or diagnostic MRI scan. Further wherein when the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated; and when the fraction (percentage) of fucosylated PSA is greater than 0.640 (64.0%), then treat the subject with a needle biopsy and do not treat with an mpMRI scan; and when the fraction (percentage) of fucosylated PSA is less than or equal to 0.640 (64.0%), then treat the subject with an mpMRI; and when the mpMRI shows a PI-RADS score of 3, 4 or 5, then treat the subject with a needle biopsy; and when the PI-RADS score is 1 or 2 do not treat the subject with a needle biopsy.

Also provided is a method of monitoring and treating a prostate cancer patient post prostate cancer removal surgery for need of additional cancer treatment, when the fraction (percentage) of total Neu5Ac on PSA that is α2-3-linked Neu5Ac is greater than 0.280 (28.0%) recommend treatment or treat the prostate cancer patient with additional cancer treatment; and when the total Neu5Ac on PSA that is α2-3-linked Neu5Ac falls between ≥0.240 (24.0%) and ≤0.280 (28.0%), perform intensified monitoring post-treatment; and wherein when the fraction (percentage) of total Neu5Ac on PSA that is α2-3-linked Neu5Ac is less than 0.240 (24.0%), then perform no monitoring post-treatment.

In all methods of the invention, one may use any suitable α2-3-linked Neu5Ac specific neuraminidase. A preferred α2-3-linked Neu5Ac specific neuraminidase is the NEUS enzyme from Streptococcus pneumonia (NanB subtype). Neuraminidase from Salmonella typhimurium LT2 also specifically removes α2-3-linked Neu5Ac, with specificity similar to NEUS, and can also be used. Human neuraminidase 2 (NEU2) can also be used.

Any suitable non-specific neuraminidase capable of removing both α2-3- and α2-6-linked Neu5Ac maybe used. A preferred non-specific neuraminidase capable of removing both α2-3- and α2-6-linked Neu5Ac is the NEUC enzyme from Clostridium perfringens (NanI subtype).

### Sequence Listing

The instant application contains a Sequence Listing which has been submitted electronically in XML file format and is hereby incorporated by reference in its entirety. Said XML copy, created on Jun. 7, 2023, is named 779434-000004_SL.xml and is 2,237 bytes in size.

### BRIEF DESCRIPTION OF THE FIGURES

FIG. 1(A) provides the illustration of one dominant glycoform of PSA and the chemical structures of α2-3- and α2-6-linked (to galactose) N-acetylneuraminic acid (Neu5Ac). FIG. 1(B) shows the workflow for quantifying the relative (fractional) amount of the total Neu5Ac that is α2-3-linked Neu5Ac and is α2-6-linked Neu5Ac on PSA extracted from a blood serum or plasma sample (referred to herein as the 23siaPSA blood test): Step 1. Extraction of PSA from PCa patient blood serum or plasma by using anti-PSA antibody. Step 2. Measurement of electrospray ionization (ESI) mass spectrum of extracted PSA, extracted PSA following treatment with NEUS and extracted PSA following treatment with NEUC. The center-of-mass (CoM) of PSA (without enzymatic treatment, with treatment with NEUS and with treatment with NEUC) is determined from each mass spectrum using center-of-mass monitoring (CoMMon). Step 3. Quantification of fractional α2-3-linked Neu5Ac content in PSA (referred to herein as % α23PSA) from the measured CoMs.

FIG. 2(A) provides analysis of N-glycans released from standard PSA (commercial) and labeled with 2-AB by HILIC-HPLC. FIG. 2(B) provides analysis of N-glycans released from standard PSA (commercial) treated with NEUS and labeled with 2-AB by HILIC-HPLC. FIG. 2(C) shows the relative abundance of all sialylated (containing Neu5Ac) N-glycans and the α2-3-linked Neu5Ac N-glycans based on HILIC-HPLC analysis. The relative abundance of all sialylated N-glycans was normalized to abundance of all N-glycans and the relative abundance of α2-3-linked Neu5Ac N-glycans was normalized to abundance of all sialylated N-glycans.

FIG. 3(A) shows the fractional abundance of total Neu5Ac that is α2-3-linked Neu5Ac in PSA (indicated as % α23PSA) from GG1 (low risk, green dot), GG2 (intermediate risk, orange dot) and GG3-5 (high risk, red dot) PCa serum samples. Error bars represent 95% confidence interval (95% CI). A fraction threshold of 0.280 is used for determining additional testing of the patient. FIG. 3(B) provides the Receiver Operating Curves (ROC) for the 23siaPSA blood test. When using 0.280 as the %23PSA test threshold for clinically significant PCa (GG2 to GG5), various ROCs are generated for distinguishing low-risk PCa (GG1) patients versus high-risk PCa patients (GG3-5) (light blue curve, AUC=0.905), for distinguishing low-risk PCa patients versus intermediate-risk (GG2) and high-risk PCa patients (red curve, AUC=0.875), for distinguishing low-risk PCa patients versus intermediate-risk PCa patients (brown curve, AUC=0.792), and for distinguishing intermediate-risk PCa patients versus high-risk PCa patients (purple curve, AUC=0.681), Detailed ROC analysis results are shown in Table D.

FIG. 4 provides information on PSA protein standards (23PSA, 26PSA and their monofucosylated analogs).

FIG. 5 provides the amino acid sequence of PSA (SEQ ID NO:1).

FIG. 6 shows the structure of CUPRA substrates (a) CS_{6SL}, (b) CS_{3SL} and (c) CS_{3SLNAc}. Also shown in (c) are the hydrolysis products resulting from treatment of CS_{3SLNAc} with NEUS or NEUC.

FIG. 7 provides representative ESI mass spectra of purified (commercial) PSA (top), α2-3 disialylated PSA (23PSA, middle) and α2-6 disialylated PSA (26PSA, bottom). Insets show the relative (to all sialylated PSA N-glycans) abundances of the two major glycoforms of PSA.

FIG. 8 shows that the invention can reduce false negatives and shows the threshold for determining whether a patient should receive additional testing, such as a needle biopsy.

FIG. 9(A) and FIG. 9(B) provide a diagram of a treatment decision tree. FIG. 9(A) shows the decision to proceed to prostate needle biopsy when the % α23PSA is >0.280 (>28.0%) and the decision to forego or avoid imaging and a prostate needle biopsy when the % α23PSA is <0.240 (<24.0%). FIG. 9(B) shows a part of the decision tree for when % α23PSA is between ≥0.240 (≥24.0%) and ≤0.280 (<28.0%). In this event, if the fraction (percentage) of fucosylated PSA (% fucoPSA) is >0.640 (>64.0%), then the subject should proceed to a prostate needle biopsy. However, if the % fucoPSA is ≤0.640 (≤64.0%), then the patient should be recommended an mpMRI scan. When the mpMRI yields a PI-RADS score of 3, 4 or 5, then the patient should be recommended a prostate needle biopsy and if the PI-RADS score is 1 or 2 then the patient should be recommended no prostate needle biopsy.

FIGS. 10(A) and 10(B) show the recovery efficiency of PSA extraction from blood serum (77.8%±2.9%) determined by adding purified PSA into 1 mL ammonia acetate (200 mM, pH 6.7, 25° C.) with a final concentration of 1.99 μg/mL (measured by the BCA protein assay). Recovery efficiency (from three replicates) was determined based on the concentration of extracted PSA determined by the BCA protein assay. (a) Calibration curve obtained by the BCA assay for quantification of PSA. (b) Concentration quantified before (red) and after extraction (orange), errors are one standard deviation from three replicates.

FIGS. 11(A) through 11(E) show the quantification of NEUS specificity using PSA standards (23PSA and 26PSA) and CUPRA substrates (CS_{3SLNAc} and CS_{6SL}, see detailed structures in FIG. 6). FIG. 11(A) provides representative ESI mass spectra acquired for aqueous ammonium acetate solutions (200 mM, pH 6.7 and 25° C.) containing NEUS (0.18 μM), ^{Uni}P_{proxy} (hCA, 5 μM), CS_{3SLNAc} (5 μM), CS_{6SL} (5 μM) and 23PSA (5 μM)) at reaction time 3 and 90 min. FIG. 11(B) provides representative ESI mass spectra acquired for aqueous ammonium acetate solutions (200 mM, pH 6.7 and 25° C.) containing NEUS (0.18 μM), ^{Uni}P_{proxy} (hCA, 5 μM), CS_{3SLNAc} (5 μM), CS_{6SL} (5 μM) and 26PSA (5 μM) at reaction time 3 and 90 min. FIG. 11(C) shows progress curves (fractional abundance of product) for CS_{3SLNAc}, CS_{6SL}, 23PSA and 26PSA. FIG. 11(D) provides the determination of initial rates by linear fitting of the first 4 data points (from reaction time 3 to 6 min). FIG. 11(E) shows the relative initial rate (normalized to that of CS_{3SLNAc}) of CS_{3SLNAc}, CS_{6SL}, 23PSA and 26PSA.

FIG. 12 shows representative ESI mass spectra of standard PSA (commercial, 5 μM) and asialo-PSA (5 μM, prepared by treating PSA with NEUC to remove all Neu5Ac).

FIGS. 13(A) and 13(B) provide the results of control experiments. FIG. 13(A) shows representative ESI mass spectra acquired for an aqueous ammonium acetate solution (200 mM, pH 6.7 and 25° C.) of NEUS (˜0.18 μM), ^{Uni}P_{proxy} (hCA, 5 μM), CS_{3SL} (12.5 μM) and ¹³C₃-Neu5Ac (internal standard (IS), 15 μM) measured at 3 min, 15 min and 90 min. FIG. 13(B) shows the time-dependent concentration of enzyme reaction product (CP_{Lac}, which is equivalent to 2,7-anhydro-Neu5Ac concentration) constructed by CUPRA-ZYME (black circle) and IS (red circle).

FIG. 14(A) shows the enzyme (NEUS) reaction progress curves (fractional abundance of product) for PSA determined by CoMMon (red circles) and using ¹³C₃-Neu5Ac as an IS (black circles). FIG. 14(B) shows the comparison of % α23PSA determined by CoMMon and IS method.

FIG. 15A, FIG. 15B, FIG. 15C, FIG. 15D and FIG. 15E are one table spanning 5 pages and it provides the assignment of N-glycans released from standard PSA (commercial), labelled with 2-AB and analyzed by HILIC-HPLC coupled with fluorescence and ESI-MS detectors. N-glycans and their structures are shown in order of increasing retention time (RT). Three replicates of relative abundances were measured.

FIG. 16 shows the ROC curve for PSA level when using a threshold of 4 ng/mL for detecting clinically significant prostate cancer (Gleason Group 2 and above).

FIG. 17 shows representative ESI mass spectra acquired for an aqueous ammonium acetate solution (200 mM, pH 6.7 and 25° C.) containing PSA extracted from blood serum incubated with NEUS for 3 min, 30 min and 180 min. Signal corresponding to the most abundant protein in blood serum, serum albumin (35-50 mg/mL in blood serum), was detected. However, the top-down native MS assay is insensitive to the presence of serum albumin as only PSA signal is considered in the CoMMon analysis.

### DETAILED DESCRIPTION OF THE INVENTION

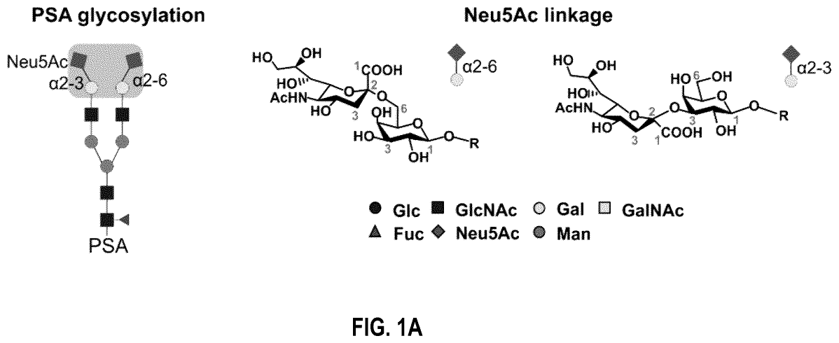

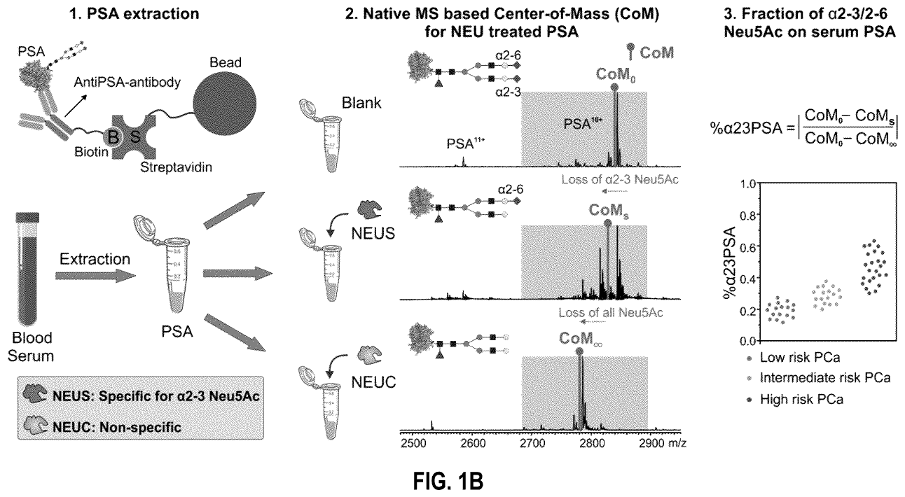

Embodiments of the invention provide a robust top-down native mass spectrometry (MS) approach, performed using a combination of specific (for α2-3-linked Neu5Ac) and non-specific neuraminidases, and employing center-of-mass (CoM) monitoring (referred herein as CoMMon) for the quantification of the relative (fractional) amount of the total N-acetylneuraminic acid (Neu5Ac) that is α2-3-linked N-acetylneuraminic acid (α2-3-linked Neu5Ac) and is α2-6-linked N-acetylneuraminic acid (α2-6-linked Neu5Ac) present on PSA extracted from blood serum or plasma (see FIG. 1). The methods disclosed herein are free of errors associated with lectin-based assays and avoid sample handling steps required for HPLC analysis. Assays of the invention were validated using purified PSA from a commercial source and PSA standards containing all α2-3- or α2-6-linked Neu5Ac. The fractional α2-3-linked Neu5Ac content (referred herein as % α23PSA) of purified PSA from a commercial source measured with the assay agrees (<2%) with values obtained by HPLC analysis of released N-glycans (see Table A and Table B).

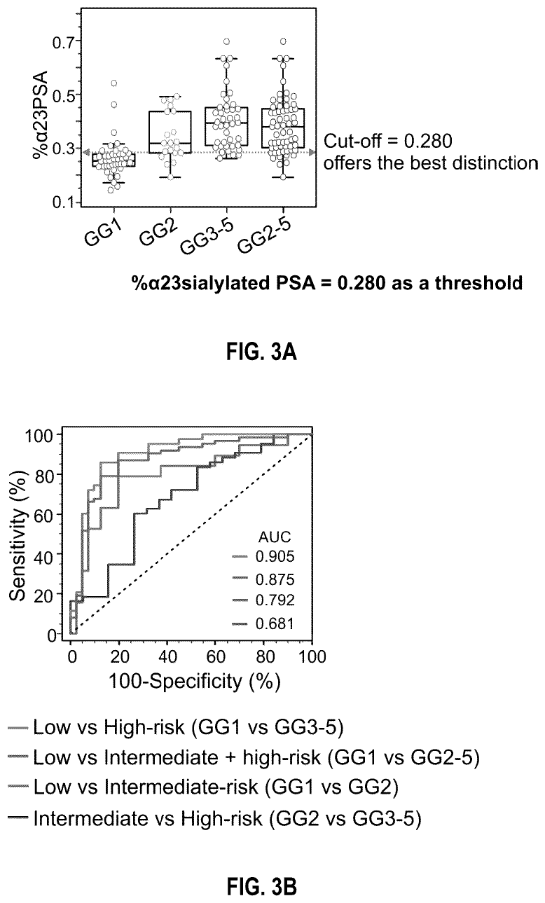

To illustrate the potential of the assay for clinical diagnosis of PCa and disease staging, the % α23PSA of PSA extracted from blood serum of low-risk (Gleason Group/GG1), intermediate-risk (GG2) and high-risk (GG3,4,5) PCa individuals were determined. A high sensitivity (85.5%) was observed as well as specificity (84.6%) for discrimination of clinically significant PCa (GG2-5) when using a % α23PSA test cut-off of 0.280 (28.0%) (area under the curve (AUC)=0.875, 0.799-0.950, 95% CI, N=102) (see FIG. 3). Together these results demonstrate that the native MS approach is a reliable and quantitative method to measure the fractional amount of the total Neu5Ac that is α2-3-linked Neu5Ac in PSA present in patient blood serum or plasma samples.

The inventors have demonstrated that top-down native MS performed using NEUS and NEUC enables the facile quantification of % α23PSA of PSA isolated from blood serum or plasma. Because of the near absolute specificity of NEUS towards α2-3-linked Neu5Ac (NEUS cleaves α2-3-linked Neu5Ac more than 350 times faster than α2-6-linked Neu5Ac), the relative α2-3- and α2-6-linked Neu5Ac content in PSA can be accurately determined from the differences in the CoM of PSA without enzymatic treatment, PSA after treatment with NEUS and PSA after treatment with NEUC, as determined from the ESI mass spectra. The specificity of the enzymes was tested and determined using the PSA standards of the invention.

Other neuraminidases with specificity for α2-3-linked Neu5Ac such as human neuraminidase 2 (NEU2) and neuraminidase from Salmonella typhimurium LT2 can be used instead of NEUS.

Notably, this top-down native MS approach is robust (insensitive to PSA contamination by co-extracted glycoproteins) and rapid (in comparison to bottom-up approaches involving the analysis of released N-glycans). Additionally, there is no requirement for calibration curves and its related standards. This is in contrast to indirect methods, such as ELISA, which rely on an immunoreactive agent to inform the investigator of the relative α2-3- and α2-6-linked Neu5Ac content in PSA. Absolute quantification of % α23PSA is a clear strength of the assay, which leads to the discrimination of clinically significant PCa (GG2-5) disease from low-risk PCa. This was previously not achieved with other top-down or bottom-up based approaches.^{8,13,27,39}

ELISA and MS methods of quantitating relative α2-3-linked Neu5Ac content of blood serum PSA as reported Yoneyama et al., relied on a % α23PSA cut-off of ˜41.5% (˜0.415) and did not find discrimination between low-risk PCa and clinically significant PCa.²⁴ Their reported AUC of 0.748 was for clinically significant PCa but had a specificity (true negative rate) of 43.4% when using a sensitivity (true positive rate) of 90%. In contrast, the dual neuraminidase-assisted top-down native MS approach described herein produced superior performance test characteristics (AUC=0.875, 0.799 to 0.950, 95% CI) with a sensitivity of 85.5% and specificity of 84.6% when using a % α23PSA of 0.280 (28.0%) for clinically significant PCa.

The dual neuraminidase-assisted top-down native MS assay of the invention shows significant promise for identifying patients with clinically significant PCa, histologically known as GG2-5 PCa disease. This has important clinical implications for patients on active surveillance. Active surveillance is a therapy option for men with low-risk PCa, wherein radical therapy (prostatectomy, radiation therapy) is deferred until their low-risk tumor (GG1) has evolved or upgraded to an intermediate/high-risk (GG2-5) form. This requires these patients to be biopsied annually or every two years to detect any tumor upgrading (from GG1 to GG2-5). However, it has been established that active surveillance patients have a 5-year cancer mortality rate <0.01% and only 30% of these patients will ever upgrade.^{40,41} This means that they are more likely to die from a needle biopsy than the low-risk cancer itself. A non-invasive means of identifying GG2-5 disease in active surveillance patients is a clinically unmet need in urology in order to minimize unnecessary needle biopsies. The histological grading of tissue biopsies is not infallible either; there exists a 10-20% biopsy sampling error for GG1 PCa when comparing the histologically-assessed needle biopsy tissue against the histologically assessed prostate in its entirety. This sample error is due to the needle missing tumors in the prostate as some of these tumors may reside on the farthest perimeter of the prostate and hence farthest away from the entry points of the 12 needles.⁴² Hence, a near perfect correlation of any biomarker to tissue biopsy-dependent diagnosis of clinically significant PCa (GG2-5) is not possible and should be re-evaluated. This intrinsic limitation of the needle biopsy procedure explains, in part, why the invention's specificity and sensitivity test characteristics are 84-86%.

The use of accurately determined % α23PSA to differentiate patients with PCa from other groups appears promising and, with more extended validation, could serve as a valuable PCa diagnosing tool. Additionally, the dual-neuraminidase native MS approach could serve as a general approach to quantifying the relative α2-3-linked Neu5Ac content of other glycoproteins related to various diseases, such as al-acid glycoprotein (diagnostic marker of lung and laryngeal cancers) and α-fetoprotein (serological marker in the diagnosis of hepatocellular cancer).⁴³⁻⁴⁵

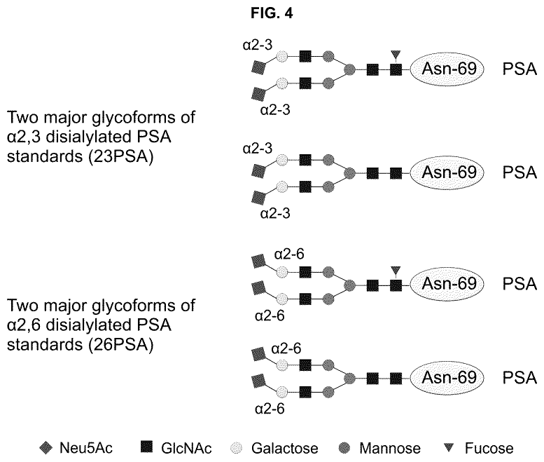

An embodiment of the invention includes an α2-3 disialylated PSA (23PSA) protein standard and an α2-6 disialylated PSA (26PSA) protein standard that are useful for various methods disclosed herein (FIG. 4). The 23PSA standard is a form of PSA that contains a biantennary disialylated N-glycan (fucosylated and non-fucosylated) in which both sialic acids (Neu5Ac) have α2-3 linkages. The 26PSA standard is a form of PSA that contains a biantennary disialylated. N-glycan (fucosylated and non-fucosylated) in which both sialic acids (Neu5Ac) have α2-6 linkages (FIG. 4). The two standards were prepared from purified PSA obtained from a commercial source (Lee Biosolutions, MO, USA). The amino acid sequence of the PSA sample is given in FIG. 5. The protocols used to prepare the standards are described herein below in Example 1. As noted above, these standards were used to determine the specificity of the enzymes use in the methods of the invention.

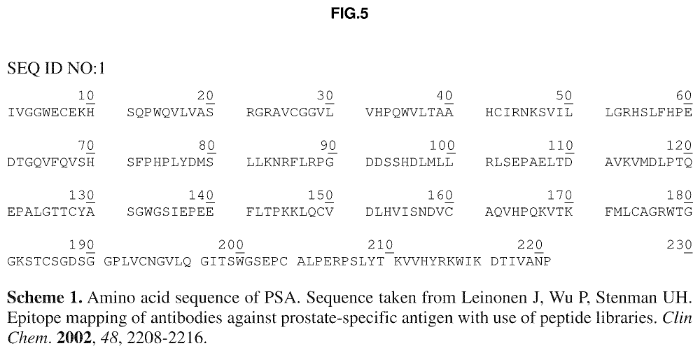

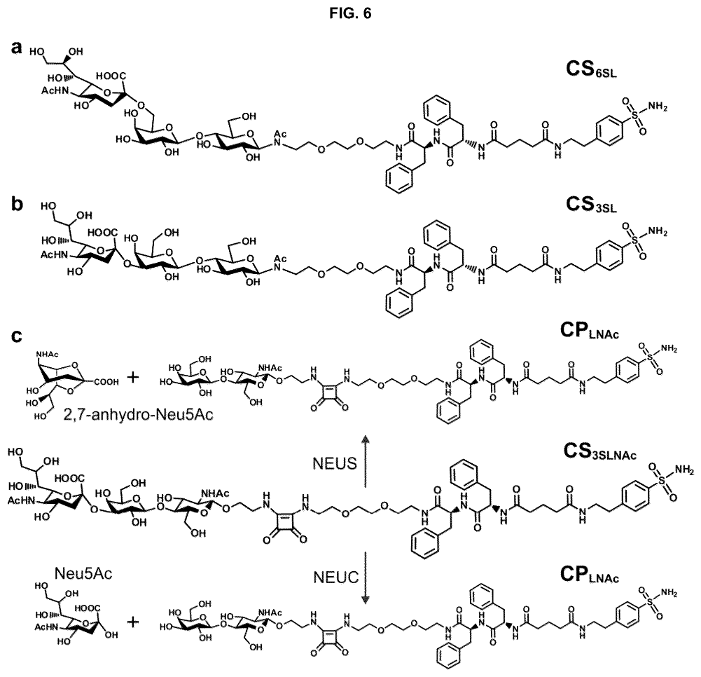

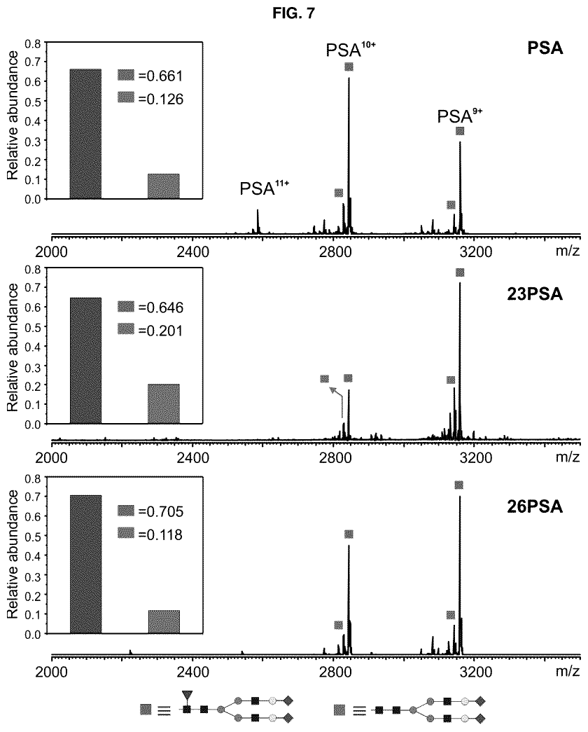

The 23PSA and 26PSA standards, combined with two modified oligosaccharide substrates (referred as to CUPRA substrates, see structure in FIG. 6), are used to test the specificity of the NEUS used in the blood serum or plasma test. NEUS must catalyze only the hydrolysis of α2-3-linked Neu5Ac on PSA. Linkage-specific hydrolysis is critical to the accuracy of the test, as any activity for α2-6-linked Neu5Ac will lead to an overestimation of the fraction of α2-3-linked Neu5Ac. Quantification of specificity is accomplished by treating mixtures of CUPR substrates, ^{Uni}P_{proxy} and 23PSA or 26PSA with NEUS and continuously monitoring the enzymatic reaction by ESI-MS. Enzyme progress curves are constructed from the time-dependent mass spectra and initial rates for hydrolysis of 23PSA and 26PSA are determined. A threshold ratio of 23PSA to 26PSA initial rates of >350 is required under the reaction conditions used. See also FIG. 7, which shows representative mass spectra of purified (commercial) PSA (top), 23PSA (middle) and 26PSA (bottom). The insets show the relative (to all sialylated PSA N-glycans) abundances of the two major glycoforms of PSA.

A further embodiment of the invention provides a method for measuring the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample. This involves extracting PSA from the blood serum or plasma sample using an anti-PSA antibody. An enzymatic treatment is performed on a first portion of the extracted PSA with an α2-3-linked Neu5Ac specific neuraminidase that specifically removes α2-3-linked Neu5Ac to obtain a first PSA extract. Then an enzymatic treatment is performed on a second portion of the extracted PSA with a non-specific neuraminidase capable of removing both α2-3- and α2-6-linked Neu5Ac to obtain a second PSA extract.

In all the methods of the invention, this step of enzymatic treatment protocol can be performed as described above, where the sample is taken from the subject and then divided into 3 portions: 1) the control where no enzymatic treatment is performed; 2) a portion that is treated with an α2-3-linked Neu5Ac specific neuraminidase that specifically removes α2-3-linked Neu5Ac; and 3) a portion that is treated with a non-specific neuraminidase capable of removing both α2-3- and α2-6-linked Neu5Ac to obtain a second PSA extract. The electrospray ionization mass spectrometry (ESI-MS) and center-of-mass (CoM) analysis of the PSA that is present in each of these portions is performed separately with each portion and then compared.

An alternative enzymatic treatment protocol can be used in all methods of the present invention as well. In this treatment, the sample is taken from the subject, but is not divided into 3 portions, treated and then tested. Instead the sample is tested and then treated and retested sequentially. For instance, the sample undergoes the electrospray ionization mass spectrometry (ESI-MS) and center-of-mass (CoM) analysis of the PSA present in the sample. That measurement is recorded. Then that same sample is subjected to the first enzyme treatment of α2-3-linked Neu5Ac specific neuraminidase that specifically removes α2-3-linked Neu5Ac and then undergoes the electrospray ionization mass spectrometry (ESI-MS) and center-of-mass (CoM) analysis of the PSA present in the sample. That measurement is recorded. Then the same sample is subjected to the second enzyme treatment of non-specific neuraminidase capable of removing both α2-3- and α2-6-linked Neu5Ac and then undergoes the electrospray ionization mass spectrometry (ESI-MS) and center-of-mass (CoM) analysis of the PSA present in the sample. That measurement is recorded. Then all three of the measurements are used in the methods.

Accordingly, in the methods described herein below either of the above enzymatic treatment protocols (3 test portions or one test portion, tested 3 different times) can be used in any of the methods of the invention.

After the enzymatic treatments described above, the next step is measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the following: i) a portion of the extracted PSA that was not enzymatically treated with a neuraminidase; and in ii) the first and second PSA extracts.

Or in the case where only one sample was used and tested three different times, measuring and quantifying are performed with electrospray ionization mass spectrometry (ESI-MS) and center-of-mass (CoM) analysis of the PSA that is present in the control and then again performed following the first and the second enzymatic treatments.

The methods then involve quantification of the fractional amount of α2-3-linked Neu5Ac in PSA from the CoM analysis.

In all methods of the invention one may use any suitable α2-3-linked Neu5Ac specific neuraminidase. A preferred α2-3-linked Neu5Ac specific neuraminidase is the NEUS enzyme from Streptococcus pneumonia (NanB subtype). Salmonella typhimurium LT2 also specifically removes α2-3-linked Neu5Ac (around 355 times faster than α2-6-linked Neu5Ac) and can be used. Human neuraminidase 2 (NEU2) can also be used.

Any suitable non-specific neuraminidase capable of removing both α2-3- and α2-6-linked Neu5Ac maybe used. A preferred non-specific neuraminidase capable of removing both α2-3- and α2-6-linked Neu5Ac is the NEUC enzyme from Clostridium perfringens (NanI subtype). Human neuraminidase 3 (NEU3) or the neuraminidase from Arthrobacter ureafaciens can also be used.

The invention also provides a method for quantifying the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample obtained from a human subject suspected of having prostate cancer to characterize whether the subject has a low risk (GG1), intermediate risk (GG2) or a high risk (GG3, 4 or 5) prostate cancer, the method comprising the PSA extractions and enzymatic treatment and quantification of the fractional amount of α2-3-linked Neu5Ac in PSA from the CoM analysis as described above. Then using the fractional amount of total Neu5Ac on PSA that is α2-3-linked Neu5Ac to predict the subject as having clinically significant prostate cancer (Gleason Group 2, 3, 4 and 5 also known as GG2-5); wherein when the fraction (percentage) of Neu5Ac on PSA that is α2-3-linked Neu5Ac is less than 0.240 (24.0%), then the subject has a high likelihood (>95% chance) of low risk (GG1) prostate cancer or no prostate cancer; wherein when the fraction (percentage) is equal to or above 0.240 (24.0%) and less than or equal to 0.280 (28.0%), then the subject has a low likelihood of clinically significant prostate cancer (GG2-5); and wherein when the fraction (percentage) is above 0.280 (28.0%), then the subject has a high likelihood (˜90%) of clinically significant prostate cancer (GG2-5).

Also provided is a method of determining whether a human subject suspected of having prostate cancer should receive a needle biopsy. This method comprises the PSA extractions and enzymatic treatment and quantification of the fractional amount of α2-3-linked Neu5Ac in PSA from the CoM analysis as described above. Then using the fractional amount of total Neu5Ac on PSA that is α2-3-linked Neu5Ac to determine whether the subject should receive a biopsy as outlined below:
i. when the fraction (percentage) of total Neu5Ac on PSA that is α2-3-linked Neu5Ac is greater than 0.280 (28.0%) recommend the subject proceed immediately to a needle biopsy and forego the need for a diagnostic MRI scan; and
ii. when the fraction (percentage) is less than 0.240 (24.0%) recommend no diagnostic MRI scan and no needle biopsy.

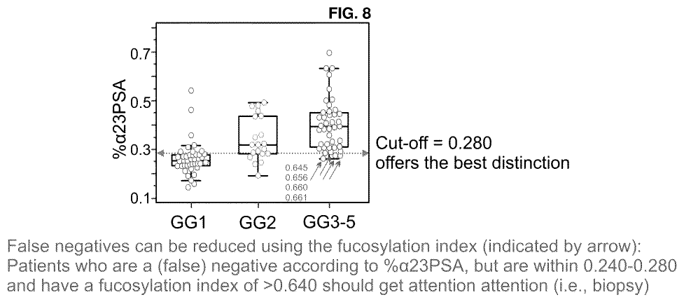

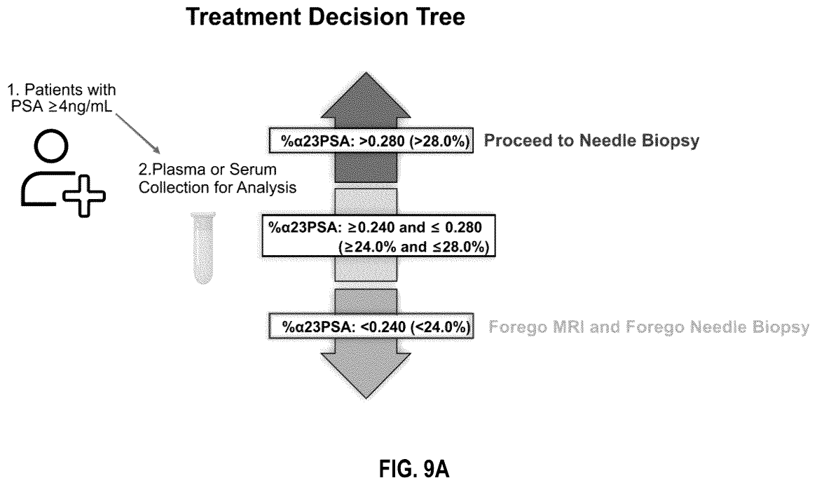

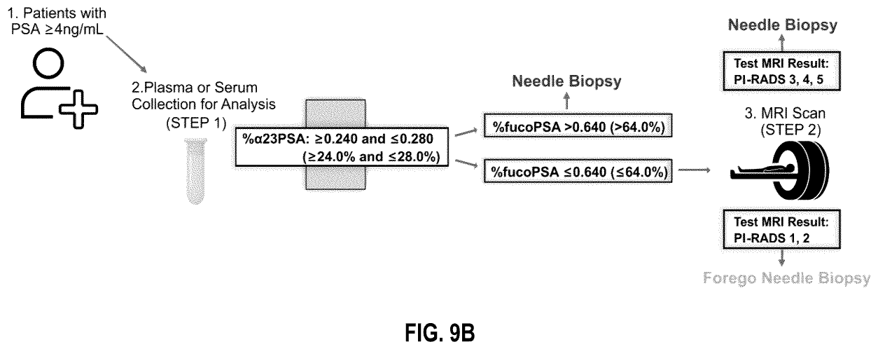

Methods described above can further comprise additional tests. When the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated (FIG. 8 and FIG. 9); and if the fraction (percentage) of fucosylated PSA is greater than 0.640 (64.0%), then recommend the subject have a needle biopsy and recommend to forego an mpMRI scan; and if the fraction (percentage) of fucosylated PSA is less than or equal to 0.640 (64.0%), then recommend the subject have an mpMRI; and when the mpMRI shows a PI-RADS score of 3, 4 or 5, then recommend a needle biopsy and if the PI-RADS score is 1 or 2 then recommend no needle biopsy (FIG. 9).

Also provided are methods of providing a diagnostic treatment (which is useful for the diagnosing of a subject suspected of having prostate cancer) such as a needle biopsy, the method comprises the PSA extractions and enzymatic treatment and quantification of the fractional amount of α2-3-linked Neu5Ac in PSA from the CoM analysis as described above. Then using the fractional amount of total Neu5Ac on PSA that is α2-3-linked Neu5Ac to determine whether the subject should receive a biopsy as outlined below:
i. when the fraction (percentage) of total Neu5Ac on PSA that is α2-3-linked Neu5Ac is greater than 0.280 (28.0%) treat the subject with a needle biopsy and forego a diagnostic Mill scan; and
ii. when the fraction (percentage) is less than 0.240 (24.0%) do not treat subject with a needle biopsy or diagnostic MRI scan.

In addition, wherein when the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated; and
when the fraction (percentage) of fucosylated PSA is greater than 0.640 (64.0%), then treat the subject with a needle biopsy and do not treat with an mpMRI scan; and
when the fraction (percentage) of fucosylated PSA is less than or equal to 0.640 (64.0%), then treat the subject with an mpMRI; and
when the mpMRI shows a PI-RADS score of 3, 4 or 5, then treat the subject with a needle biopsy; and
when the PI-RADS score is 1 or 2 do not treat the subject with a needle biopsy.

Herein is provided a method of monitoring and treating a prostate cancer patient post prostate cancer removal surgery for need of additional cancer treatment. The method comprises the PSA extractions and enzymatic treatment and quantification of the fractional amount of α2-3-linked Neu5Ac in PSA from the CoM analysis as described above. Then using the fractional amount of total Neu5Ac on PSA that is α2-3-linked Neu5Ac to determine whether the subject should be treated as outlined below. When the fraction (percentage) of total Neu5Ac on PSA that is α2-3-linked Neu5Ac is greater than 0.280 (28.0%) recommend or treat the prostate cancer patient with additional cancer treatment; and when the total Neu5Ac on PSA that is α2-3-linked Neu5Ac falls between ≥0.240 (24.0%) and ≤0.280 (28.0%), perform intensified monitoring post-treatment; and wherein when the fraction (percentage) of total Neu5Ac on PSA that is α2-3-linked Neu5Ac is less than 0.240 (24.0%), then no perform no monitoring post-treatment.

The invention also provides an α2-3 disialylated PSA protein standard (23PSA) (that has been manually modified enzymatically—see Example 3) having the amino acid sequence set forth in SEQ ID NO: 1 and an α2-6 disialylated PSA protein standard (26PSA) having the amino acid sequence set forth in SEQ ID NO: 1 (which was also manually modified enzymatically—see Example 3).

### EXAMPLES

### Example 1: Materials and Methods

### Proteins, Enzymes, Substrates and Other Reagents.

Prostate specific antigen (PSA, MW 28,430 Da, purified from human seminal plasma) was purchased from LEE Biosolutions (Maryland Heights, MO). Human carbonic anhydrase (hCA, type I, MW 28,848 Da) and neuraminidase from Clostridium perfringens (NanI subtype, denoted as NEUC) were purchased from Sigma-Aldrich Canada (Oakville, Canada). Neuraminidase from Streptococcus pneumonia (NanB subtype, denoted as NEUS) was purchased from New England Biolabs (Beverly, MA, USA). Asialo-PSA was prepared by incubating PSA with NEUC in 200 mM ammonium acetate (pH 6.7) at room temperature overnight. Asialo-PSA was further used to produce α2-3 or α2-6 disialylated PSA by incubating with CMP-Neu5Ac and ST3Gal4 or ST6Gal1, respectively. All proteins and enzymes were dialyzed against an aqueous solution of 200 mM ammonium acetate (pH 6.7) using an Amicon 0.5 mL micro concentrator (EMD Millipore, Billerica, MA) with a MW cutoff of 3 kDa and stored at −20° C. until used. The concentrations of protein and enzyme stock solutions were measured by UV absorption at 280 nm.

Neu5Acα2-6Galβ1-4Glc (6SL) was purchased from Carbosynth (San Diego, CA), Neu5Acα2-3Galβ1-4Glc (3SL) was purchased from Elicityl SA (Crolles, France) and Neu5Acα2-3Galβ1-4GlcNAc-ethylamine (3SLNAc-ethylamine) was synthesized, as described elsewhere.^{46,47} N-acetyl-neuraminic acid-1,2,3-¹³C₃ (Neu5Ac-¹³C₃) was purchased from Omicron Biochemicals Inc. (South Bend, IN, USA). Cytidine-5′-monophosphate-N-acetylneuraminic acid (CMP-Neu5Ac) were purchased from Sigma-Aldrich Canada (Oakville, Canada). CUPRA substrates CS_{3SLNAc} and CS_{6SL} (see detail structure in FIG. 6) were synthesized as described elsewhere.^{46,47}

### Sample Collection.

Blood serum samples from 102 patients were collected in Sunnybrook Research Institute with the approval of the REB committee at Sunnybrook Health Sciences Centre (Toronto, ON). Whole blood from patients were collected into blood serum-specific vacutainers (BD Biosciences Inc.) and then allowed to clot overnight. After centrifugation at 1000×g's for 15 minutes at room temperature, the blood serum upper phase supernatant was aliquoted and stored at −80° C. until used.

### PSA Extraction.

PSA from blood serum was extracted using anti-PSA-antibody based on previously reported protocols (FIG. 1B).^{27,37,38}

### Hydrophilic Interaction-Ultra High Performance Liquid Chromatography (HILIC) Analysis of N-Glycans

N-glycans present on PSA were released enzymatically, isolated as a mixture and labeled with 2-aminobenzamide (2-AB), and then analyzed by hydrophilic interaction-ultra high performance liquid chromatography (HILIC) on a Thermo Scientific™ Vanquish™ UHPLC system coupled with fluorescence (Thermo Scientific, Waltham, MA, USA) and ESI-MS detectors (Thermo Q Exactive Orbitrap).

### Mass Spectrometry.

All ESI-MS measurements were carried out in positive ion mode using a Q Exactive Ultra High Mass Range (UHMR) Hybrid Quadrupole-Orbitrap or a Q Exactive Quadrupole Orbitrap mass spectrometer (Thermo Fisher Scientific, Bremen, Germany). Both instruments were equipped with a modified nanoflow ESI (nanoESI) source.

### NEUS Substrate Specificity.

The specificity of the NEUS enzyme for PSA N-glycans with α2-3- and α2-6-linked Neu5Ac was quantified using the CUPRA-ZYME assay performed using the CUPRA substrates CS_{3SLNAc} and CS_{6SL} (see FIG. 6). Details of the assay and data analysis procedures are described elsewhere. 46

### Statistical Analysis.

The relative (fractional) amount of total Neu5Ac that is present as α2-3-linked Neu5Ac (denoted as % α23PSA) on PSA extracted from blood serum samples from low-risk (GG1), intermediate-risk (GG2), and high-risk (GG3,4,5) PCa patients was calculated using Eq 1 (see Example 2). The mean % α23PSA and the corresponding 95% confidence interval values were calculated for each group. Receiver operating characteristics (ROC) curves were generated based on % α23PSA for distinguishing between low-risk (GG1) and clinically significant (GG2-5) disease, and then between low-risk and high-risk (GG3-5) disease. Youden's J-index analysis was used to generate the % α23PSA cut-offs for each comparison. Intergroup differences were statistically analyzed by a Student's t-test for normally distributed variables. In all statistical analyses, the significance level was set at p<0.05 and assuming no normality distributions.

### Example 2: Results and Discussion

### Assay Overview and Validation.

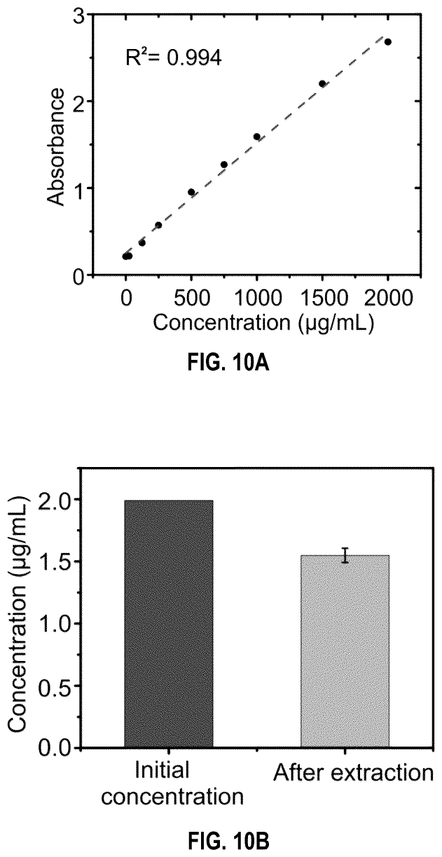

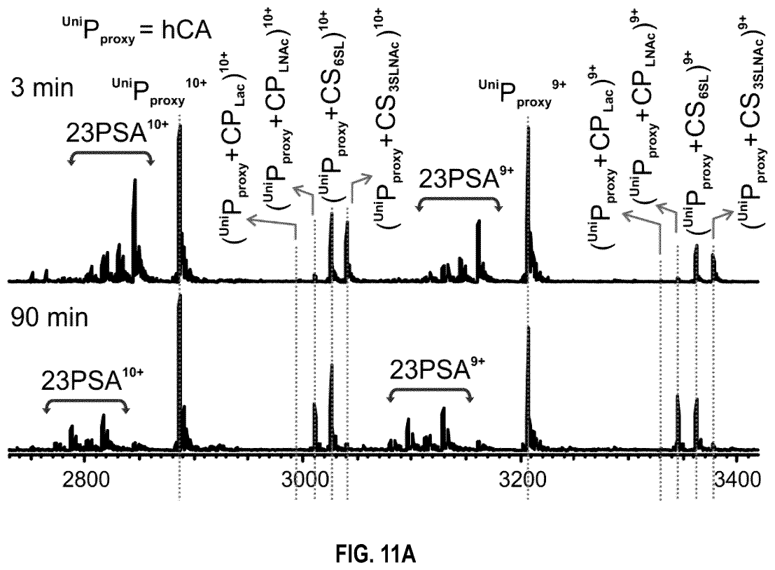

An overview of the workflow for the top-down native MS method for quantifying the % α23PSA of PSA from blood serum or plasma samples, extracted using anti-PSA antibody, is provided in FIG. 1 (with a recovery rate of 77.8%±2.9%, FIG. 10). The assay is based on changes in the center-of-mass (CoM) of intact PSA (as measured by native MS and CoMMon)⁴⁷ upon treatment with a α2-3-linked Neu5Ac specific neuraminidase and by treatment with a non-specific neuraminidase (which can remove both α2-3- and α2-6-linked Neu5Ac). NEUS enzyme from Streptococcus pneumonia (NanB subtype) was used to selectively remove α2-3-linked Neu5Ac;^{48,49} while the NEUC enzyme, a neuraminidase from Clostridium perfringens (NanI subtype), served as the non-specific neuraminidase to remove all Neu5Ac (both α2-3-linked and α2-6-linked Neu5Ac on the Asn-69 of PSA protein).⁴⁸ The fraction of α2-3-linked Neu5Ac (% α23PSA) was calculated as the ratio of the CoM change upon treatment with NEUS to the CoM change resulting from treatment with NEUC (Eq 1). The CoM of extracted PSA prior to enzyme treatment is denoted as CoM₀. The CoM of extracted PSA after treatment with NEUS is denoted as CoMs. The CoM of extracted PSA after treatment with NEUC is denoted as CoM_{∞}.

% α23PSA=|(CoM₀−CoMs)/(CoM₀−CoM_{∞})| (Eq 1)

Differences in the Neu5Ac linkage specificities of NEUC and NEUS enzymes are key to the performance of our newly developed assay. As shown by our laboratory and others, NEUC catalyzes the hydrolysis of both α2-3- and α2-6-linked Neu5Ac, albeit with a preference for α2-3-linked Neu5Ac.^{47,50} In contrast, NEUS is highly specific for glycans with α2-3-linked Neu5Ac.^{48,49} However, its relative activity towards α2-6-linked Neu5Ac in PSA had not been definitively established. We assessed the specificity of NEUS for α2-3- and α2-6-linked Neu5Ac using CUPRA substrates (CS_{3SLNAc} and CS_{6SL}) and PSA standards containing all α2-3-linked Neu5Ac (23PSA) or all α2-6-linked Neu5Ac (26PSA). The accuracy of the % α23PSA values measured by the top-down native MS approach with CoMMon was assessed by comparison with the results of Neu5Ac content obtained using an internal standard (IS) approach and from a bottom-up LC-MS approach, wherein N-glycans were released from PSA before or after enzyme treatment with NEUS or NEUC, labeled with 2-aminobenzamide (2-AB) and analyzed by HILIC-HPLC.

### NEUS Specificity for α2-3- and α2-6-Linked Neu5Ac.

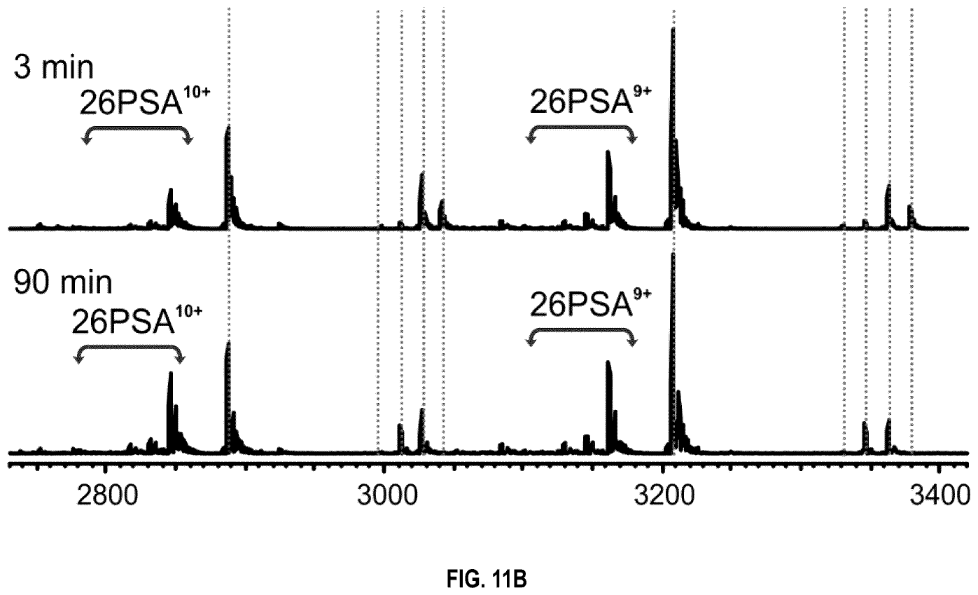

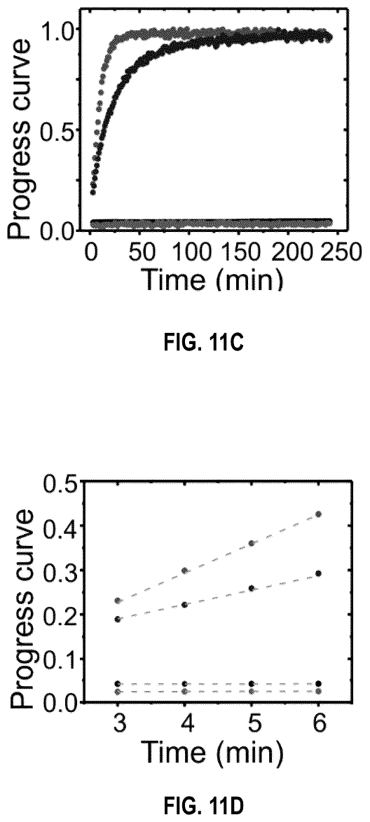

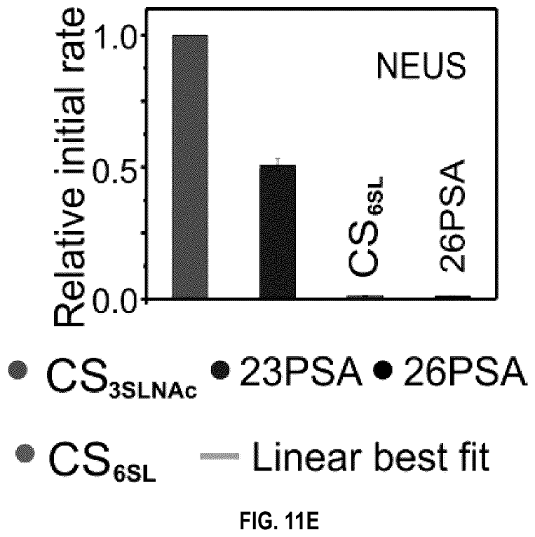

To determine the relative activity of NEUS for α2-3- and α2-6-linked Neu5Ac, native MS analysis with CoMMon was performed on the 23PSA and 26PSA standards of the present invention. These measurements were carried out in the presence of CS_{3SLNAc} and CS_{6SL} (see structures in FIG. 6), and using hCA as the ^{Uni}P_{proxy}. Time-resolved mass spectra were acquired continuously at reaction times (t) from 3 min (the minimum time required to load the tip and initiate spray) to 90 min. Shown in FIG. 11 are the representative ESI mass spectra measured for aqueous ammonium acetate solutions (200 mM, pH 6.7 and 25° C.) of NEUS, CS_{6SL}, CS_{3SLNAc}, ^{Uni}P_{proxy} with 23PSA (FIG. 11A) or 26PSA (FIG. 11B).

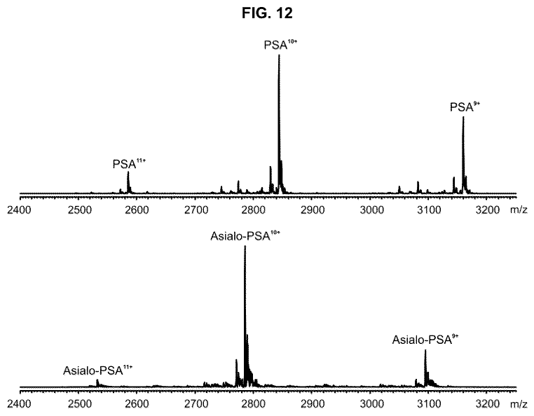

NEUS enzyme specificity for α2-3- and α2-6-linked Neu5Ac N-glycans was quantified using the reaction progress curves (FIG. 11C). For the CS (CS_{3SLNAc} and CS_{6SL}), the (fractional) reaction progress (denoted as f_{CS,t}) at a given reaction time was calculated from the measured relative abundance (Ab) of the corresponding gas-phase ions of ^{Uni}P_{proxy} bound to CS (^{Uni}P_{proxy}+CP) and CP (^{Uni}P_{proxy}+CP) and the initial concentrations, Eqs 2-4. For 23PSA and 26PSA, the fractional reaction progress (denoted as f t) curves were constructed using the CoMMon method and Eqs 5-7. The initial and final CoM values (CoM₀ and CoM_{∞}, respectively) were determined from the mass spectra of PSA and asialo-PSA (FIG. 12). As expected, only CS_{3SLNAc} and 23PSA underwent appreciable hydrolysis in the presence of NEUS. Based on relative (normalized to CS_{3SLNAc}) initial rates (FIGS. 11C, 11D and 11E, calculated by Eq 8), NEUS-catalyzed hydrolysis of CS_{3SLNAc} is 290-fold faster than CS_{6SL}; 23PSA hydrolysis is 360-fold faster than 26PSA. According to these data, the use of NEUS to effect complete removal of α2-3-linked Neu5Ac from PSA will result in the fractional loss of <0.002 of the α2-6-linked Neu5Ac, which falls within three standard deviations from the mean (0.0024).

$${[ CP ]}_{t} = \frac{{[ CS ]}_{0} {Ab}_{t} {(}^{Uni} {P}_{proxy} + CP )}{{Ab}_{t} {(}^{Uni} {P}_{proxy} + CS ) + {Ab}_{t} {(}^{Uni} {P}_{proxy} + CP )} \qquad ( Eq 2 )$$

$${[ CS ]}_{t} = \frac{{[ CS ]}_{0} {Ab}_{t} {(}^{Uni} {P}_{proxy} + CS )}{{Ab}_{t} {(}^{Uni} {P}_{proxy} + CS ) + {Ab}_{t} {(}^{Uni} {P}_{proxy} + CP )} \qquad ( Eq 3 )$$

$${f}_{CS {,}_{t}} = \frac{{[ CS ]}_{t}}{{[ CS ]}_{t} + {[ CP ]}_{t}} \qquad ( Eq 4 )$$

$${f}_{t} = \vert ( {CoM}_{0} - {CoM}_{t} ) / ( {CoM}_{0} - {CoM}_{∞} ) \vert \qquad ( Eq 5 )$$

$${CoM}_{t} = {\sum }_{i} {f}_{i , t} {MW}_{i , t} \qquad ( Eq 6 )$$

$${f}_{i , t} = \frac{{\sum }_{n} {{Ab}_{i} ( {MW}_{i , t} )}^{n +}}{{\sum }_{i} {\sum }_{n} {{Ab}_{i} ( {MW}_{i , t} )}^{n +}} \qquad ( Eq 7 )$$

$${v}_{rel} = \frac{{v}_{0 , PSA}}{{v}_{0 , CS}} \qquad ( Eq 8 )$$

Accordingly, the various methods of the invention can use any neuraminidase enzyme specific for α2-3-linked Neu5Ac. Preferably this enzyme must remove α2-3-linked Neu5Ac at least 350 times faster than removal of α2-6-linked Neu5Ac. The preceding paragraphs set forth methods of determining this activity. In some embodiments this enzyme is NEUS enzyme from Streptococcus pneumonia (NanB subtype).

The various methods of the invention can use any non-specific neuraminidase enzyme that is capable of removing both α2-3- and α2-6-linked Neu5Ac. Preferably this enzyme must completely cleave off all of the α2-3-linked and α2-6-linked Neu5Ac within the given time set for the reaction. For example, if the enzymatic reaction was to be run for 15 minutes, the non-specific neuraminidase enzyme would completely cleave off all α2-3-linked and α2-6-linked Neu5Ac within 15 minutes. Alternatively, the skilled artisan would just allow the enzymatic reaction to proceed as long as was needed to allow the enzyme to complete the cleavage of all α2-3-linked and α2-6-linked Neu5Ac. It is of course preferred to use a non-specific neuraminidase enzyme that can complete the cleavages in a shorter amount of time. In some embodiments the non-specific neuraminidase capable of removing both α2-3- and α2-6-linked Neu5Ac is NEUC enzyme from Clostridium perfringens (NanI subtype).

### Comparison of α2-3 Neu5Ac Content Measured by Top-Down, Bottom Up and IS MS Methods.

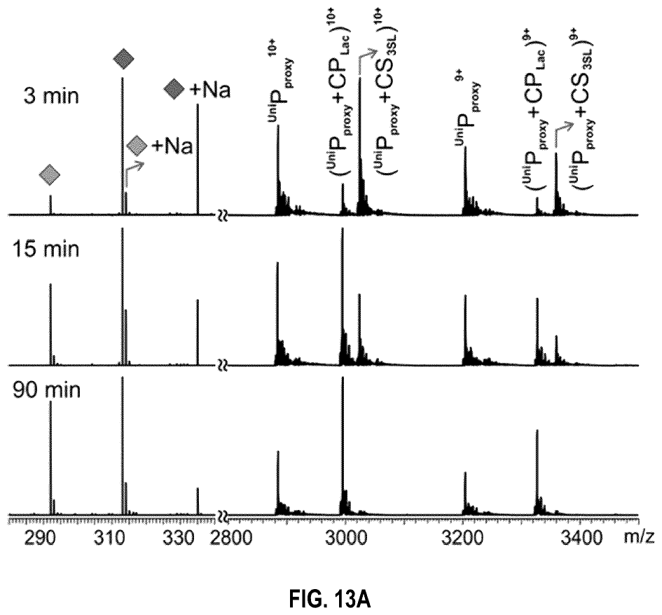

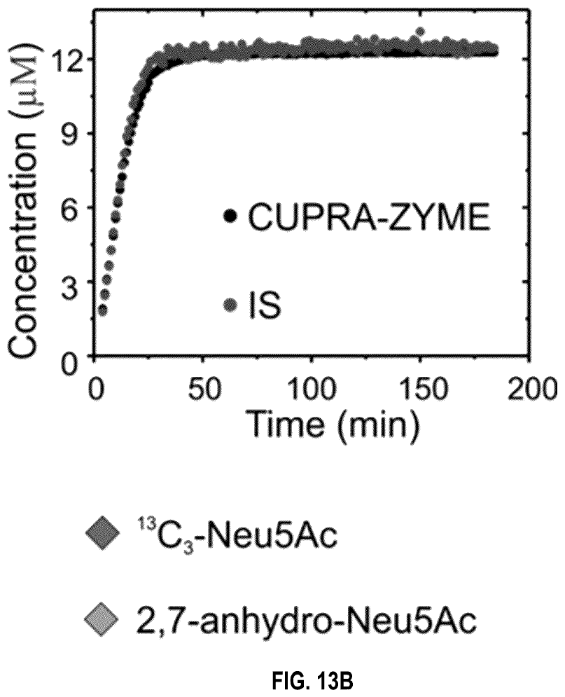

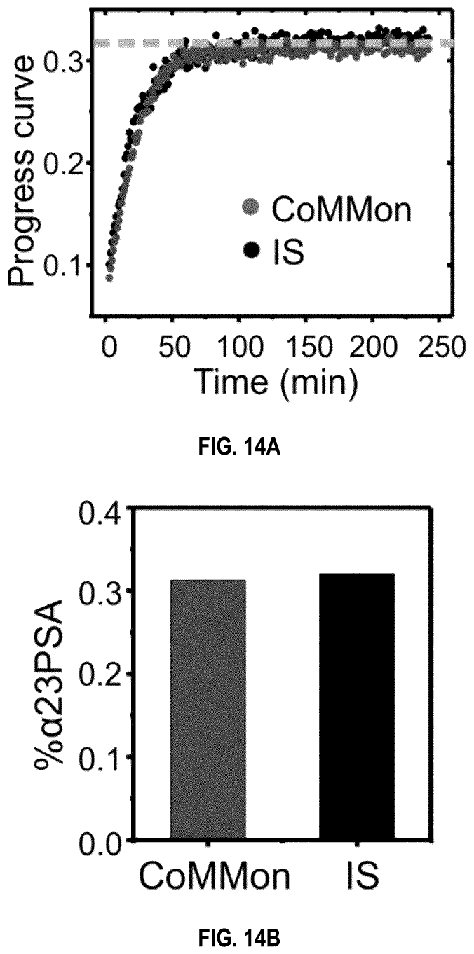

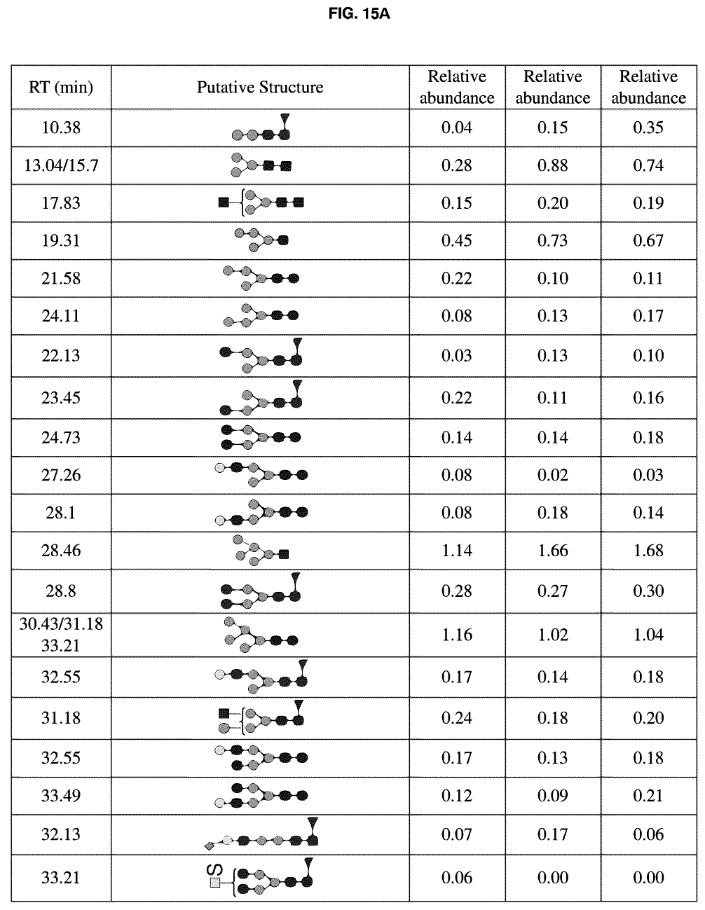

To demonstrate the reliability of the top-down native MS assay for quantifying the relative amount of α2-3-linked Neu5Ac on PSA, hydrolysis kinetics measurements were performed on the commercial PSA sample in the presence of ¹³C₃-Neu5Ac, which served as an internal standard (IS). Unlike most neuraminidases, NEUS produces the 2,7-anhydro-Neu5Ac product, rather than Neu5Ac, from substrates containing α2-3-linked Neu5Ac (FIG. 6 and FIG. 13a). However, according to the results of control experiments (FIG. 13B), the ESI-MS response factors of ¹³C₃-Neu5Ac and 2,7-anhydro-Neu5Ac are indistinguishable (within experimental error). Notably, the progress curves for the NEUS-catalyzed hydrolysis of a commercial PSA sample measured by CoMMon and the IS method (Eq 9) are indistinguishable (FIG. 14) and reach limiting fractional values of % α23PSA that are in agreement (0.3164±0.0024, top-down native MS; 0.3205±0.0012, IS approach, Table A).

$${f}_{2 , 7 anhydro - Neu 5 Ac} = \frac{Ab ( 2 , 7 anhydro - Neu 5 Ac ) + Ab ( 2 , 7 anhydro - Neu 5 {Ac}^{Na} )}{Ab ( Neu 5 Ac {-}^{13} {C}_{3} ) + Ab ( Neu 5 Ac {-}^{13} {C}_{3}^{Na} )} \qquad ( Eq 9 )$$

TABLE A
Relative (fractional) amount of Neu5Ac that is α2-3linkedNeu5Ac
(relative to all sialylated N-glycans) in a commercial PSA sample
determined by top-down native MS and IS approaches.^{a}

| Method | Relative amount of α2-3-linked Neu5Ac |
|---|---|
| top-down native MS | 0.3164 ± 0.0024 |
| IS | 0.3205 ± 0.0012 |

^{a}Error corresponds to three standard deviations; measurements performed in triplicates.

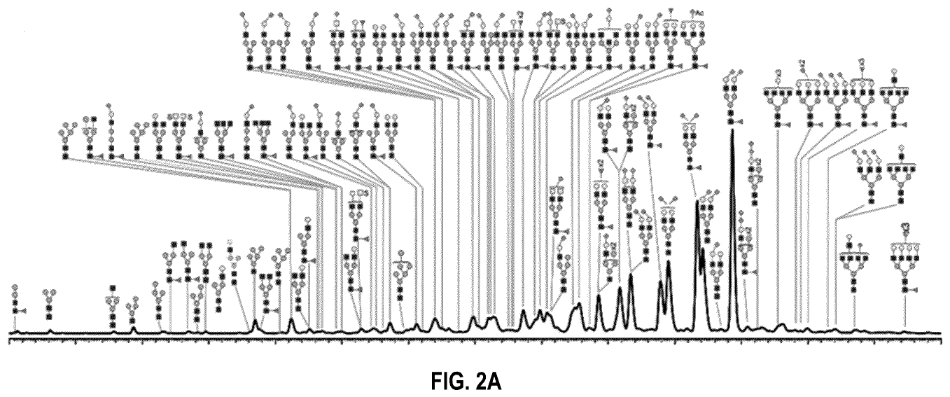

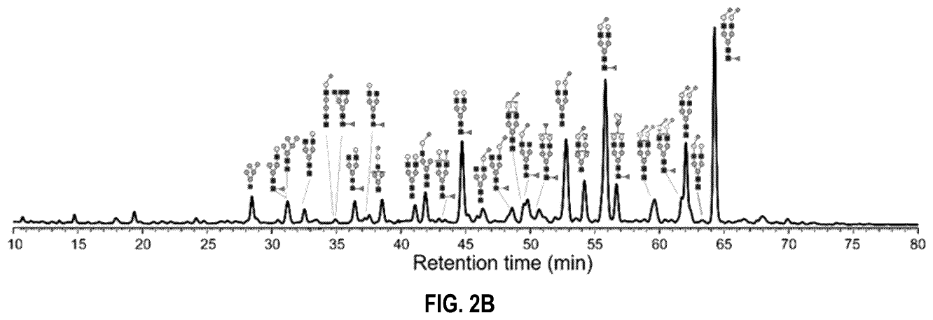

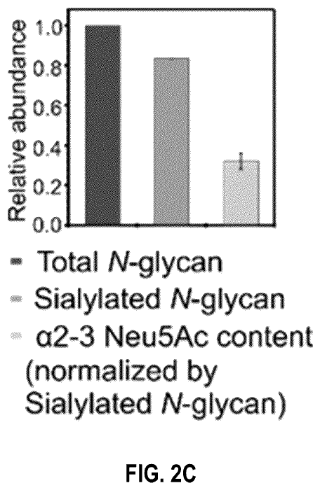

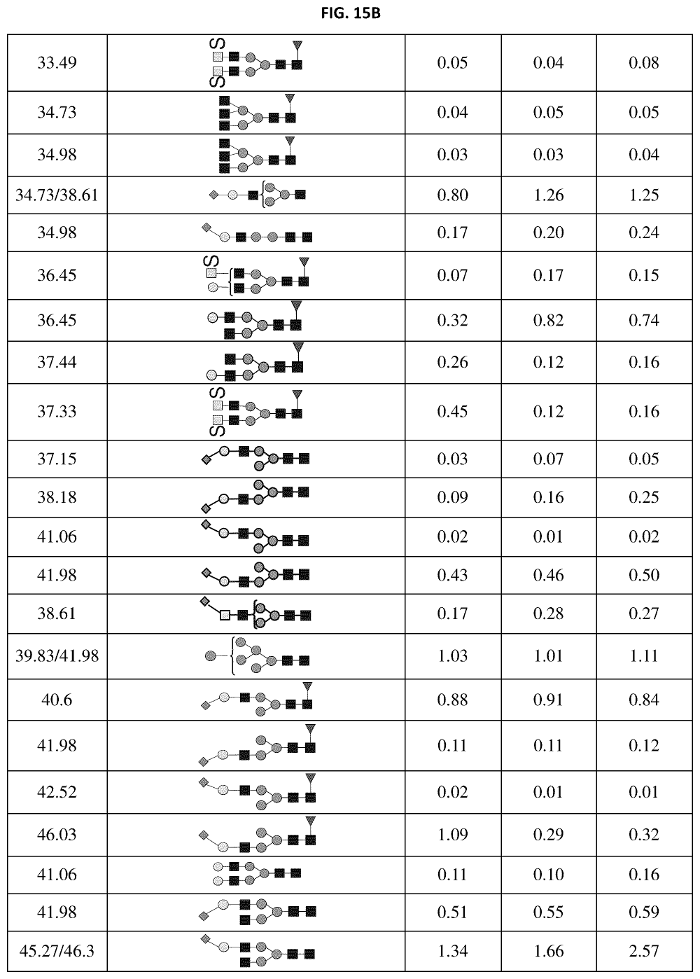

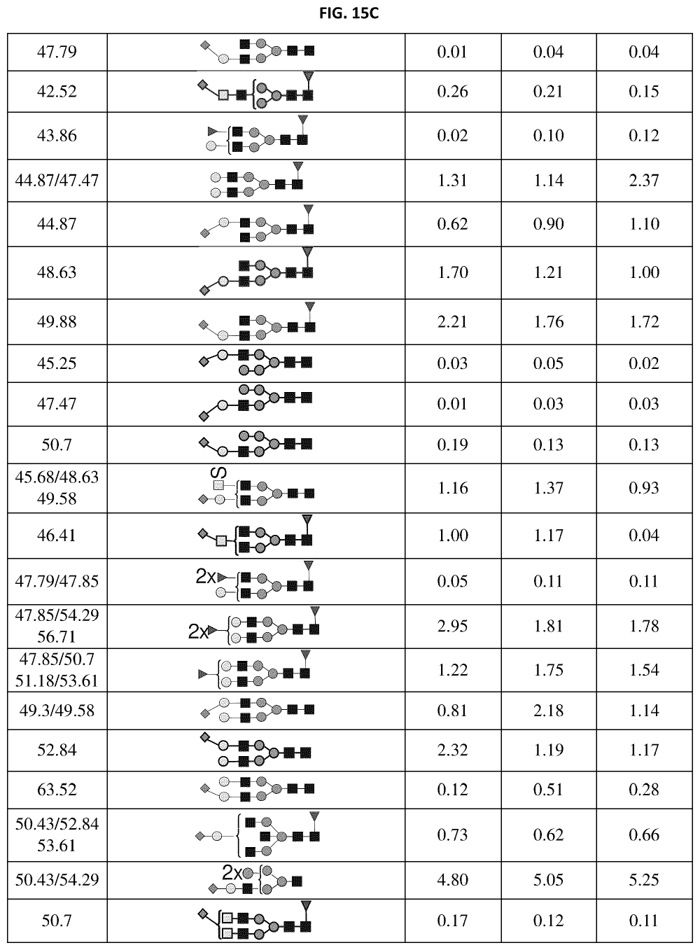

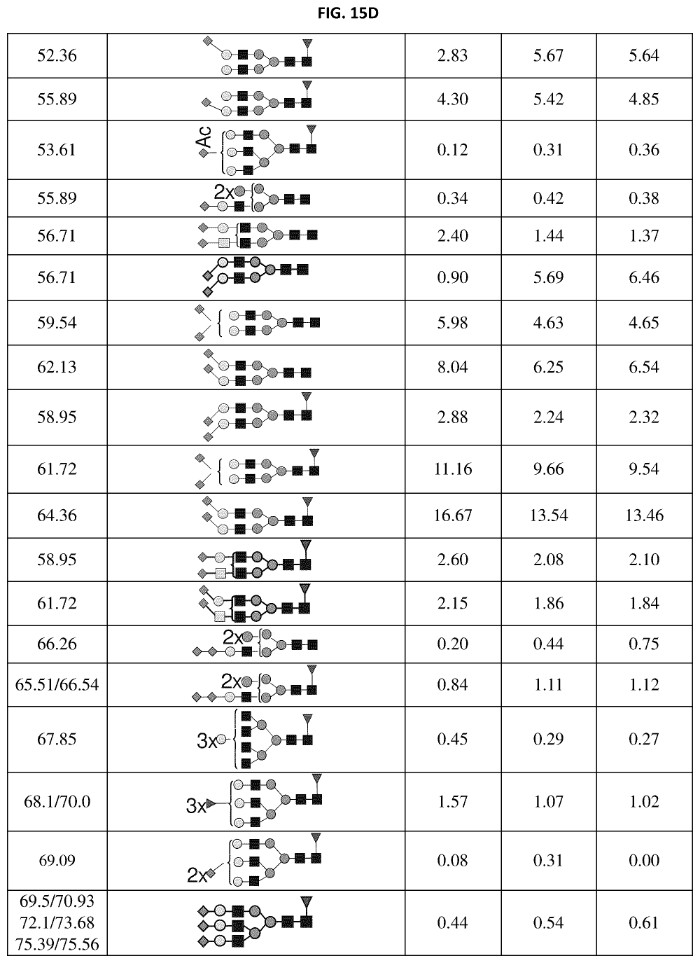

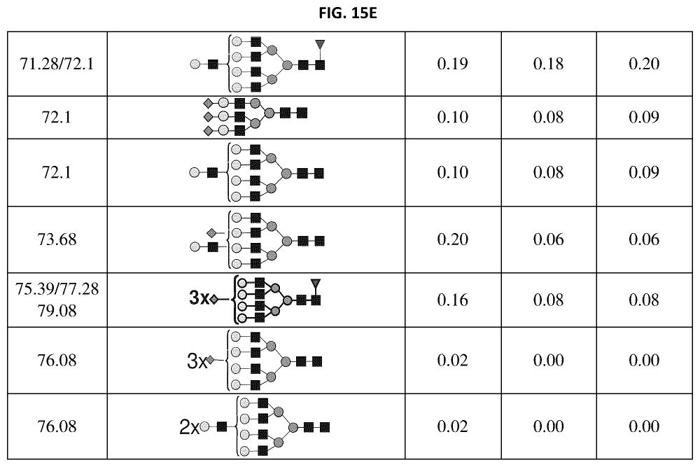

The relative amount of α2-3 Neu5Ac in the commercial PSA sample was also quantified using a bottom-up LC-MS approach, wherein N-glycans released from the PSA sample, before and after treatment with NEUS, were labeled with a fluorophore (2-AB) and analyzed by HILIC-HPLC coupled with fluorescence and ESI-MS detection. Analysis identified 85 distinct PSA N-glycans (FIG. 2 and FIG. 15A-E), corresponding predominantly to fucosylated bi-antennary disialylated N-glycans ((31.37±2.89)%). Tri- and tetra-antennary complex-type N-glycans were also present, although at low abundances ((2.90±0.12)% and (0.84±0.21)%, respectively), in agreement with previously published results.^{51,52} Of the 85 N-glycans, 49 are sialylated, containing from one to three Neu5Ac residues; sialylated N-glycans contribute (83.6±0.6)% to total abundance of the detected N-glycans. The relative amount of α2-3-linkedNeu5Ac in the PSA sample, found from analysis of the N-glycans before and after treatment with NEUS, was 0.322±0.1203 (FIG. 2 C and Table B).

TABLE B
Relative abundance of PSA N-glycans containing Neu5Ac
(sialic acid) and N-glycans containing α2-3-linked
Neu5Ac determined by HILIC-HPLC analysis.^{a}

| | Relative abundance |
|---|---|
| Total N-glycans | 1 |
| N-glycans containing | 0.8364 ± 0.0063 |
| Neu5Ac | (relative to Total N-glycans) |
| Neu5Ac-containing N- | 0.3221 ± 0.1203 |
| glycans corresponding to | (relative to N-glycans containing Neu5Ac) |
| α2-3-linked Neu5Ac | |

^{a}Error corresponds to three standard deviations; measurements performed in triplicates.

This value agrees with the values obtained by the top-down native MS and IS methods. Together, the results of these analyses demonstrate that our top-down native MS assay can accurately quantify the relative (fractional) amount of Neu5Ac that is α2-3-linked Neu5Ac (% α23PSA) on PSA.

### Quantification of α2-3 Neu5Ac in Blood Serum PSA for Discrimination of Low-Risk (GG1), Intermediate-Risk (GG2), and High-Risk (GG3/4/5) PCa.

Following analytical validation of the top-down native MS method for quantifying the % α23PSA of PSA, the assay was applied to PSA extracted from blood serum samples obtained from PCa patients representing all risk types; patient cohort clinical information is provided in Table C.

TABLE C
Patient Clinical Information.

| | | | Positive Cores | | | |
|---|---|---|---|---|---|---|
| | | Gleason | No. | | | |
| | | Group | Positive | | Patients | PSA level |
| Risk type | N (%) | (1-5) | Cores | | (%) | (ng/mL) |
| Low-risk | 41 | GG1 | 1-2 | | 16 (40%) | 7.68 ± 1.74 |
| | (40%) | | 3-4 | | 14 (35%) | 6.49 ± 0.81 |
| | | | ≥5 | | 9 (23%) | 7.80 ± 0.73 |
| | | | Not deter- | | 2 (5%) | 0.41, 10.50 |
| | | | mined | | Average | 6.95 ± 0.79 |
| Interme- | 20 | GG2 | 1-2 | Cores | 4 (25%) | 12.71 ± 5.63 |
| diate-risk | (20%) | | 3-4 | Cores | 7 (35%) | 8.29 ± 2.67 |
| | | | ≥5 | Cores | 9 (45%) | 6.24 ± 1.03 |
| | | | | | Average | 8.25 ± 1.52 |
| High-risk | 20 | GG3 | 1-2 | Cores | 2 (10%) | 9.81, 14.0 |
| | (20%) | | | | | |
| | 19 | GG4 | 3-4 | Cores | 5 (25%) | 9.85 ± 3.00 |
| | (19%) | | ≥5 | Cores | 13 (65%) | 16.74 ± 4.08^{a} |
| | | | | | Average | 14.41 ± 2.74 |
| | | | 1-4 | Cores | 2 (11%) | 6.0, 13.17 |
| | | | ≥5 | Cores | 17 (89%) | 28.25 ± 8.20 |
| | | | | | Average | 26.28 ± 7.25 |
| | 2 | GG5 | ≥5 | Cores | 2 (100%) | 17.3, 87.6 |
| | (2%) | | | | Average | 21.95 ± 4.18 |

^{a}A denoted outlier of PSA = 1436 ng/mL was removed.

Native mass spectra of the untreated extracted PSA were used for calculation of CoM₀. Native mass spectra of extracted PSA following incubation with NEUS to remove all α2-3-linked Neu5Ac were used to calculate CoMs, and native mass spectra of extracted PSA following incubation NEUC to remove all Neu5Ac residues from PSA were used to determine CoM_{∞}. The relative amounts of α2-3-linked Neu5Ac in blood serum PSA was calculated using Eq 1.

The % α23PSA measured for PSA extracted from blood serum samples representing GG1-5 PCa patients are shown in FIG. 3A. The data reveal an upper boundary maximum of 0.317 (31.7%) of α2-3-linked Neu5Ac (0.256±0.069, 0.233 to 0.278, 95% CI; N=41) in low-risk PCa (GG1) blood serum samples, consistent with other published data.²⁷ A significant increase in % α23PSA was found in intermediate-risk (GG2) (0.319±0.088, 0.282 to 0.416, 95% CI; N=20, p<0.001) and high-risk (GG3-5) (0.394±0.107, 0.308 to 0.454, 95% CI; N=41) PCa patient blood serum samples. These results are novel and clinically relevant in that this assay can discriminate GG1 (low-risk) disease from GG2 (intermediate-risk) disease, which has not been previously achieved and a clinical need. Youden's J-index analysis of the receiver operating characteristic (ROC) curves in FIG. 3B determined that a % α23PSA cut-off of 0.280 (28.0%) led to test sensitivity and specificity values of 85.5% and 84.6%, respectively (Table D) for identifying clinically significant PCa (GG2-5).

TABLE D
Result of ROC curves for low vs high risk (GG1 vs GG3-5), low vs
Intermediate & High risk (GG1 vs GG2-5), low vs intermediate risk
(GG1 vs GG2) and intermediate vs high risk (GG2 vs GG3-5).

| | Sensitivity | Specificity | | |
|---|---|---|---|---|
| | (%) | (%) | AUC | Criterion |
| Low vs High risk | 86.0 | 87.5 | 0.905 | 0.291 |
| (GG1 vs GG3-5) | | | | |
| Low vs Intermediate & | 85.5 | 84.6 | 0.875 | 0.280 |
| High risk (GG1 vs GG2-5) | | | | |
| Low vs Intermediate risk | 78.9 | 80.0 | 0.792 | 0.280 |
| (GG1 vs GG2) | | | | |
| Intermediate vs High risk | 60.5 | 73.7 | 0.681 | 0.360 |
| (GG2 vs GG3-5) | | | | |

This resulted in an AUC=0.792±0.064, 0.682 to 0.894, 95% CI (this ROC analysis, only includes data on GG1 versus GG2), for identifying only intermediate-risk PCa (GG2, sensitivity of 78.9% and specificity of 80.0%) and an AUC=0.875±0.038, 0.799 to 0.950 95% CI for identifying clinically significant PCa (GG2-5, sensitivity and specificity of 85.5% and 84.6% respectively). While not clinically relevant, applying the same cut-off produced an AUC=0.905 when only identifying high-risk PCa (GG3-5). As anticipated, there was moderate sensitivity and specificity for discrimination between intermediate (GG2) and high-risk (GG3-5) PCa with an AUC of 0.681; having strong discrimination between GG2 and GG3-5 is not clinically relevant.

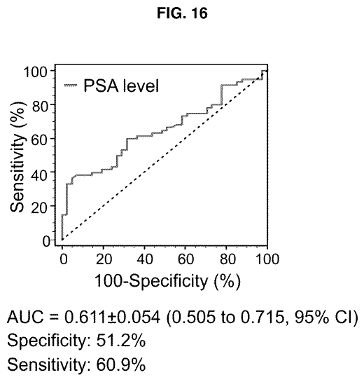

In contrast, blood serum PSA levels as measured clinically via ELISA did not offer clinical benefit for distinguishing clinically significant PCa from low-risk PCa. In FIG. 16, serum PSA led to an AUC=0.611±0.054 (0.5049 to 0.7160, 95% CI) with a sensitivity of 60.94% and specificity of 51.16% when using a PSA threshold of >6.72 ng/mL. This is consistent with the literature in that blood serum PSA levels do not distinguish between low-risk and clinically significant PCa. Several other diagnostic biomarkers have been developed to improve the prediction of clinically significant PCa. Some of these include the Prostate Health Index (PHI), the four-kallikrein panel (4Kscore), and the ExoDx IntelliScore.^{8,9,53} Recent reviews of these methods concluded that, while all these tests have clinical utility, they do not add substantially in diagnostic value and are relatively costly to perform. 10,11 For example, the AUCs for each of these tests are 0.67,⁵⁴ 0.652,⁵⁵ and 0.73,⁵⁶ respectively. In contrast, the % α23PSA assay would enable clinical laboratories to reduce unnecessary biopsies by 85% if implemented in the diagnostic pathway of localized PCa.

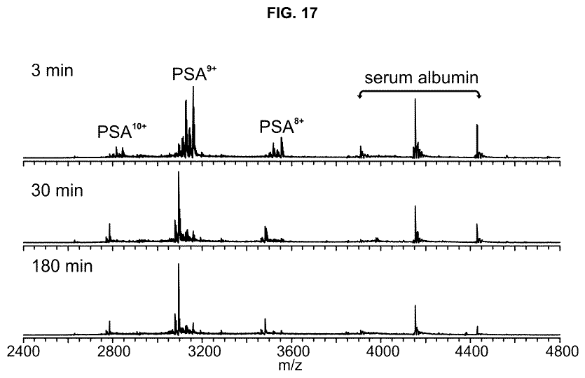

Hence, unprecedented high sensitivity and specificity for clinically significant PCa made possible by the two-enzymes approach utilized by embodiments of the invention, combined with top-down native MS, has strong clinical implications for improving the diagnostic pathway of PCa. This MS-based assay can be readily applied to monitor the relative α2-3-linked Neu5Ac content of extracted PSA for clinical diagnosis of PCa and may help to reduce the need for prostate needle biopsy procedures, a potentially lethal procedure that should be withheld from patients who harbor low-risk PCa (GG1) disease. It should be also noted that all of PSA samples extracted from blood serum were contaminated by blood serum albumin, which is the most abundant protein in blood serum (35-50 mg/mL). Remaining blood serum albumin can be a source of error for the quantification of α2-3-linked Neu5Ac content in lectin-based or bottom-up methods due to glycosylated albumin in blood serum.⁵⁷ In contrast, such error is eliminated in the top-down native MS assay, because the PSA ions selected for data analysis by CoMMon do not overlap with albumin ions (FIG. 17).

### Example 3: Protocol for Preparing α2-3 and α2-6 Disialylated Human PSA Standards (23PSA and 26PSA)

### Materials:

Human prostate specific antigen (PSA, from LEE Biosolutions), neuraminidase from Clostridium perfringens (denoted as NEUC, NanI subtype, Sigma Aldrich), cytidine-5′-monophospho-N-acetylneuraminic acid (CMP-Neu5Ac, Sigma Aldrich), MnCl₂ (Sigma Aldrich), Amicon Ultra-0.5 Centrifugal Filter Unit (size 3 kDa, Sigma Aldrich). The human sialyltransferase ST3Gal4 (Uniprot P15907, amino acid residues 75-406) and human sialyltransferase ST6Gal1 (Uniprot Q11206, amino acid residues 41-333), provided by Prof. Kelley Moremen (University of Georgia), were expressed in Freestyle 293F cells (Thermo Fisher Scientific) as green fluorescent protein (GFP) fusions in the pGEn2 vector as previously described. 58

### 1. Preparation of Asialo-PSA:

Step 1: Mix 100 μL of PSA (1.5 mg/mL) with 0.5 μL of the stock solution of NEUC (˜0.5 μM). No dialysis of PSA is required.
Step 2: Incubate the mixture at room temperature (25° C.) for 48 hours to remove all sialic acid (N-acetylneuraminic acid, Neu5Ac).
Step 3: Further incubate the mixture at 45-50° C. for another 72-96 hours to fully deactivate NEUC.
Step 4: Dialyze the mixture with water using a 3 kDa filter to remove all the Neu5Ac to a final volume ˜20 μL; store the asialo-PSA at −20° C.

### 2. Preparation of α2-3 Disialylated PSA (23PSA):

Step 5: Add 30 μL (excess) CMP-Neu5Ac (1 mM) and 10 μL MnCl₂ (1 mM) to 10 μL of the asialo-PSA stock solution.
Step 6: Mix the solution with 15 μL of ST3Gal4 solution (1 mg/mL) and incubate the mixture at room temperature for 24 hours.
Step 7: Dialyze the mixture with water using a 3 kDa filter to remove all of the MnCl₂, CMP-Neu5Ac and CMP.
Step 8 (Optional): Repeat Steps 5 and 6, as needed, to achieve a high degree of PSA sialylation.
Step 9: Dialyze the mixture with water and store the 23PSA at −20° C.

### 3. Preparation of α2-6 Disialylated PSA (26PSA):

Step 10: Add 30 μL (excess) CMP-Neu5Ac (1 mM) to 10 μL of the asialo-PSA stock solution.
Step 11: Mix the solution with 15 μL of ST6Gal1 solution (1 mg/mL) and incubate the mixture solution at room temperature for 24 hours.
Step 12: Dialyze the mixture with water using a 3 kDa filter to remove all of the CMP-Neu5Ac and CMP.
Step 13 (Optional): Repeat Step 10 and 11, as needed, to achieve a high degree of PSA sialylation.
Step 14: Dialyze the mixture with water and store the 26PSA at −20° C.

### REFERENCES

(1) Siegel, R. L.; Miller, K. D.; Jemal, A. Cancer statistics, 2020. CA. Cancer J. Clin. 2020, 70, 7-30.
(2) Sung, H.; Ferlay, J.; Siegel, R. L.; Laversanne, M.; Soerjomataram, I.; Jemal, A.; Bray, F. Global cancer statistics 2020: GLOBOCAN estimates of incidence and mortality worldwide for 36 cancers in 185 countries. CA. Cancer J. Clin. 2021, 71, 209-249.
(3) Schroder, F. H.; Hugosson, J.; Roobol, M. J.; Tammela, T. L.; Ciatto, S.; Nelen, V.; Kwiatkowski, M.; Lujan, M.; Lilja, H.; Zappa, M.; Denis, L. J, Recker, F.; Berenguer, A.; Maattanen, L.; Bangma, C. H.; Aus, G.; Villers, A.; Rebillard, X.; van der Kwast, T.; Blijenberg, B. G.; Moss, S. M.; de Koning, H. J.; Auvinen, A. ERSPC Investigators. Screening and prostate-cancer mortality in a randomized european study. N. Engl. J. Med. 2009, 360, 1320-1328.
(4) Schroder, F. H.; Hugosson, J.; Roobol, M. J.; Tammela, T. L.; Zappa, M.; Nelen, V.; Kwiatkowski, M.; Lujan, M.; Maattanen, L.; Lilja, H.; Denis, L. J.; Recker, F.; Paez, A.; Bangma, C. H.; Carlsson, S.; Puliti, D.; Villers, A.; Rebillard, X.; Hakama, M.; Stenman, U. H.; Kujala, P.; Taari, K.; Aus, G.; Huber, A.; van der Kwast, T. H.; van Schaik, R. H.; de Koning, H. J.; Moss, S. M.; Auvinen, A. ERSPC Investigators. Screening and prostate cancer mortality: results of the european randomised study of screening for prostate cancer (ERSPC) at 13 years of follow-up. Lancet. 2014, 84, 2027-2035.
(5) Shoag, J. E.; Nyame, Y. A.; Gulati, R.; Etzioni, R.; Hu, J. C. Reconsidering the trade-offs of prostate cancer screening. N. Engl. J. Med. 2020, 382, 2465-2468.
(6) Etzioni, R.; Penson, D. F.; Legler, J. M.; di Tommaso, D.; Boer, R.; Gann, P. H.; Feuer, E. J. Overdiagnosis due to prostate-specific antigen screening: Lessons from U.S. prostate cancer incidence trends. JNCI J. Natl. Cancer Inst. 2002, 94, 981-990.
(7) Loeb, S.; Bjurlin, M. A.; Nicholson, J.; Tammela, T. L.; Penson, D. F.; Carter, H. B.; Carroll, P.; Etzioni, R. Overdiagnosis and overtreatment of prostate cancer. Eur. Urol. 2014, 65, 1046-1055.
(8) Brewster, D. H.; Fischbacher, C. M.; Nolan, J.; Nowell, S.; Redpath, D.; Nabi, G. Risk of hospitalization and death following prostate biopsy in Scotland. Public Health 2017, 142, 102-110.
(9) Jesus, C. M. N. de; Corrêa, L. A.; Padovani, C. R. Complications and risk factors in transrectal ultrasound-guided prostate biopsies. Sao Paulo Med. J. 2006, 124, 198-202.
(10) Loeb, S.; Vellekoop, A.; Ahmed, H. U.; Catto, J.; Emberton, M.; Nam, R.; Rosario, D. J.; Scattoni, V; Lotan, Y Systematic review of complications of prostate biopsy. Eur. Urol. 2013, 64, 876-892.
(11) Loeb, S.; Carter, H. B.; Berndt, S. I.; Ricker, W.; Schaeffer, E. M. Complications after prostate biopsy: Data from SEER-Medicare. J. Urol. 2011, 186, 1830-1834.
(12) Saini, S. PSA and beyond: Alternative prostate cancer biomarkers. Cell. Oncol. 2016, 39, 97-106.
(13) Hatano, K.; Yoneyama, T.; Hatakeyama, S.; Tomiyama, E.; Tsuchiya, M.; Nishimoto, M.; Yoshimura, K.; Miyoshi, E.; Uemura, H.; Ohyama, C.; Nonomura, N.; Fujita, K. Simultaneous analysis of serum α2,3-linked sialylation and core-type fucosylation of prostate-specific antigen for the detection of high-grade prostate cancer. Br. J. Cancer. 2022, 126, 764-770.
(14) Chang, E. K.; Gadzinski, A. J.; Nyame, Y. A. Blood and urine biomarkers in prostate cancer: Are we ready for reflex testing in men with an elevated prostate-specific antigen? Asian J. Urol. 2021, 8, 343-353.
(15) Behnken, H. N.; Ruthenbeck, A.; Schulz, J. M.; Meyer, B. Glycan analysis of prostate specific antigen (PSA) directly from the intact glycoprotein by HR-ESI/TOF-MS. J Proteome Res. 2014, 13, 997-1001.
(16) Kaluza, A.; Szczykutowicz, J.; Ferens-Sieczkowska, M. Glycosylation: Rising potential for prostate cancer evaluation. Cancers 2021. 13, 3726-3728.
(17) Sugar, S.; Toth, G.; Bugyi, F.; Vekey, K.; Kardszi, K.; Drahos, L.; Turiak, L. Alterations in protein expression and site-specific N-glycosylation of prostate cancer tissues. Sci. Rep. 2021, 11, 15886.
(18) Hatakeyama, S.; Yoneyama, T.; Tobisawa, Y.; Ohyama, C. Recent progress and perspectives on prostate cancer biomarkers. Int. J. Clin. Oncol. 2017, 22, 214-221.
(19) Pihikova, D.; Kasak, P.; Kubanikova, P.; Sokol, R.; Tkac, J. Aberrant sialylation of a prostate-specific antigen: Electrochemical label-free glycoprofiling in prostate cancer serum samples. Anal. Chico. Acta 2016, 934, 72-79.
(20) Peracaula, R.; Tabares, G.; Royle, L.; Harvey, D. J.; Dwek, R. A.; Rudd, P. M.; de Llorens, R. Altered glycosylation pattern allows the distinction between prostate-specific antigen (PSA) from normal and tumor origins. Glycobiology 2003, 13, 457-470.
(21) Ohyama, C.; Hosono, M.; Nitta, K.; Oh-eda, M.; Yoshikawa, K.; Habuchi, T.; Arai, Y; Fukuda, M. Carbohydrate structure and differential binding of prostate specific antigen to Maackia amurensis lectin between prostate cancer and benign prostate hypertrophy. Glycobiology 2004, 14, 671-679.
(22) Gilgunn, S.; Murphy, K.; Stöckmann, H.; Conroy, P. J.; Murphy, T. B.; Watson, R. W.; O'Kennedy, R. J.; Rudd, P. M.; Saldova, R. Glycosylation in indolent, significant and aggressive prostate cancer by automated high-throughput N-glycan profiling. Int. J. Mol. Sci. 2020, 21, 9233.
(23) Haga, Y.; Uemura, M.; Baba, S.; Inamura, K.; Takeuchi, K.; Nonomura, N.; Ueda, K. Identification of multisialylated LacdiNAc structures as highly prostate cancer specific glycan signatures on PSA. Anal. Chem. 2019, 91, 2247-2254.
(24) Yoneyama, T.; Ohyama, C.; Hatakeyama, S.; Narita, S.; Habuchi, T.; Koie, T.; Mori, K.; Hidari, K. I.; Yamaguchi, M.; Suzuki, T.; Tobisawa, Y. Measurement of aberrant glycosylation of prostate specific antigen can improve specificity in early detection of prostate cancer. Biochem. Biophys. Res. Commun. 2014, 448, 390-396.
(25) Saldova, R.; Fan, Y.; Fitzpatrick, J. M.; Watson, R. W. G.; Rudd, P. M. Core fucosylation and α2-3 sialylation in serum N-glycome is significantly increased in prostate cancer comparing to benign prostate hyperplasia. Glycobiology 2011, 21, 195-205.
(26) Ferrer-Batalle, M.; Llop, E.; Ramirez, M.; Aleixandre, R. N.; Saez, M.; Comet, J.; De Llorens, R.; Peracaula, R. Comparative study of blood-based biomarkers, α2,3-sialic acid PSA and PHI, for high-risk prostate cancer detection. Int. J. Mol. Sci. 2017, 18, 845.
(27) Llop, E.; Ferrer-Batallé, M.; Barrabés, S.; Guerrero, P. E.; Ramirez, M.; Saldova, R.; Rudd, P. M.; Aleixandre, R. N.; Comet, J.; de Llorens, R.; Peracaula, R. Improvement of prostate cancer diagnosis by detecting PSA glycosylation-specific changes. Theranostics 2016, 6, 1190-1204.
(28) Gratacós-Mulleras, A.; Duran, A.; Asadi Shehni, A.; Ferrer-Batallé, M.; Ramirez, M.; Comet, J.; de Llorens, R.; Saldova, R.; Llop, E.; Peracaula, R. Characterisation of the main PSA glycoforms in aggressive prostate cancer. Sci. Rep. 2020, 10, 1-14.
(29) Wang, C.; Höti, N.; Lih, T. M.; Sokoll, L. J.; Zhang, R.; Zhang, Z.; Zhang, H.; Chan, D. W. Development of a glycoproteomic strategy to detect more aggressive prostate cancer using lectin-immunoassays for serum fucosylated PSA. Clin. Proteomics. 2019, 16, 13-21.
(30) Haese, A.; Tin, A. L.; Carlsson, S. V; Sjoberg, D. D.; Pehrke, D.; Steuber, T.; Huland, H.; Graefen, M.; Scardino, P. T.; Schlomm, T.; Vickers, J. A.; Lilja, H.; Sauter, G. A pre-specified model based on four Kallikrein markers in blood improves predictions of adverse pathology and biochemical recurrence after radical prostatectomy. Br. J. Cancer 2020, 123, 604-609.
(31) Wang, W.; Wang, M.; Wang, L.; Adams, T. S.; Tian, Y.; Xu, J. Diagnostic ability of % p2PSA and prostate health index for aggressive prostate cancer: A meta-analysis. Sci. Rep. 2014, 4, 5012.
(32) van der Burgt, Y. E. M.; Siliakus, K. M.; Cobbaert, C. M.; Ruhaak, L. R. HILIC-MRM-MS for linkage-specific separation of sialylated glycopeptides to quantify prostate-specific antigen proteoforms. J. Proteome Res. 2020, 19, 2708-2716.
(33) Reider, B.; Jarvas, G.; Krenkova, J.; Guttman, A. Separation based characterization methods for the N-glycosylation analysis of prostate-specific antigen. J. Pharm. Biomed. Anal. 2021, 194, 113797.
(34) Kammeijer, G. S. M.; Jansen, B. C.; Kohler, I.; Heemskerk, A. A. M.; Mayboroda, O. A.; Hensbergen, P. J.; Schappler, J.; Wuhrer, M. Sialic acid linkage differentiation of glycopeptides using capillary electrophorsis—electrospray ionization—mass spectrometry. Sci. Rep. 2017, 7, 3733.
(35) Kammeijer, G. S. M.; Nouta, J.; de la Rosette, J. J. M. C. H.; de Reijke, T. M.; Wuhrer, M. An in-depth glycosylation assay for urinary prostate-specific antigen. Anal. Chem. 2018, 90, 4414-4421.
(36) Gabriele, C.; Prestagiacomo, L. E.; Cuda, G.; Gaspari, M. Mass spectrometry-based glycoproteomics and prostate cancer. Int. J. Mol. Sci. 2021, 22, 5222.
(37) Etxebarria, J.; Reichardt, N. C. Methods for the absolute quantification of N-glycan biomarkers. Biochim. Biophys. Acta 2016, 1860, 1676-1687.
(38) Zhou, X.; Yang, G.; Guan, F. Biological Functions and Analytical Strategies of Sialic Acids in Tumor. Cells 2020, 9, 273-291.
(39) Yoneyama, T.; Yamamoto, H.; Sutoh Yoneyama, M.; Tobisawa, Y.; Hatakeyama, S.; Narita, T.; Kodama, H.; Momota, M.; Ito, H.; Narita, S.; et al. Characteristics of α2,3-SialylN-glycosylated PSA as a biomarker for clinically significant prostate cancer in men with elevated PSA level. Prostate 2021, 81, 1411-1427.
(40) Klotz, L.; Zhang, L.; Lam, A.; Nam, R.; Mamedov, A.; Loblaw, A. Clinical results of long-term follow-up of a large, active surveillance cohort with localized prostate cancer. J. Clin. Oncol. 2010, 28, 126-131.
(41) Klotz, L.; Vesprini, D.; Sethukavalan, P.; Jethava, V; Zhang, L.; Jain, S.; Yamamoto, T.; Mamedov, A.; Loblaw, A. Long-term follow-up of a large active surveillance cohort of patients with prostate cancer. J. Clin. Oncol. 2015, 33, 272-277.
(42) Fine, Sampson W., Epstein, Jonoathan I, A contemporary study correlating prostate needle biopsy and radical prostatectomy Gleason score. J. Urol. 2008, 179, 1335-8.
(43) Uslu, C.; Taysi, S.; Akcay, F.; Sutbeyaz, M. Y; Bakan, N. Serum free and bound sialic acid and alpha-1-acid glycoprotein in patients with laryngeal cancer. Ann. Clin. Lab. Sci. 2003, 33, 156-159.
(44) Gao, Z.; Xu, M.; Yue, S.; Shan, H.; Xia, J.; Jiang, J.; Yang, S. Abnormal sialylation and fucosylation of saliva glycoproteins: Characteristics of lung cancer-specific biomarkers. Curr. Res. Pharmacol. Drug Discov. 2022, 3, 100079.
(45) Mondal, G.; Chatterjee, U.; Chawla, Y. K.; Chatterjee, B. P. Alterations of glycan branching and differential expression of sialic acid on alpha fetoprotein among hepatitis patients. Glycoconj. J. 2011, 28, 1-9.
(46) Li, Z.; Kitov, P. I.; Han, L.; Kitova, E. N.; Mozenah, F.; Rodrigues, E.; Chapla, D. G.; Moremen, K. W.; Macauley, M. S.; Klassen, J. S. CUPRA-ZYME: An assay for measuring carbohydrate-active enzyme activities, pathways, and substrate specificities. Anal. Chem., 2020, 92, 3228-3236.
(47) Li, Z.; Kitov, P. I.; Kitova, E. N.; Bui, D. T.; Moremen, K. W.; Wakarchuk, W. W.; Mahal, L. K.; Macauley, M. S.; Klassen, J. S. Quantifying carbohydrate-active enzyme activity with glycoprotein substrates using electrospray ionization mass spectrometry and center-of-mass monitoring. Anal. Chem. 2021, 93, 15262-15270.
(48) Xu, G.; Potter, J. A.; Russell, R. J. M.; Oggioni, M. R.; Andrew, P. W.; Taylor, G. L. Crystal structure of the NanB sialidase from Streptococcus Pneumoniae. J. Mol. Biol. 2008, 384, 436-449.
(49) Gut, H.; King, S. J.; Walsh, M. A. Structural and functional studies of Streptococcus Pneumoniae neuraminidase B: An intramolecular trans-sialidase. FEBS Lett. 2008, 582, 3348-3352.
(50) Newstead, S. L.; Potter, J. A.; Wilson, J. C.; Xu, G.; Chien, C.-H.; Watts, A. G.; Withers, S. G.; Taylor, G. L. The structure of Clostridium perfringens NanI sialidase and its catalytic intermediates. J. Biol. Chem. 2008, 283, 9080-9088.
(51) Ideo, H.; Kondo, J.; Nomura, T.; Nonomura, N.; Inoue, M.; Amano, J. Study of glycosylation of prostate-specific antigen secreted by cancer tissue-originated spheroids reveals new candidates for prostate cancer detection. Sci. Rep. 2020, 10, 2708-2726.
(52) Song, E.; Mayampurath, A.; Yu, C.-Y; Tang, H.; Mechref, Y. Glycoproteomics: Identifying the glycosylation of prostate specific antigen at normal and high isoelectric points by LC-MS/MS. J. Proteome Res. 2014, 13, 5570-5580.
(53) McKiernan, J.; Donovan, M. J.; Margolis, E.; Partin, A.; Carter, B.; Brown, G.; Torkler, P.; Noerholm, M.; Skog, J.; Shore, N.; Andriole, G.; Thompson, I.; Carroll, P. A prospective adaptive utility trial to validate performance of a novel urine exosome gene expression assay to predict high-grade prostate cancer in patients with prostate-specific antigen 2-10 ng/ml at initial biopsy. Eur. Urol. 2018, 74, 731-738.
(54) Wang, W.; Wang, M.; Wang, L.; Adams, T. S.; Tian, Y.; Xu, J. Diagnostic ability of % p2PSA and prostate health index for aggressive prostate cancer: A meta-analysis. Sci. Rep. 2014, 4, 5012.
(55) Haese, A.; Tin, A. L.; Carlsson, S. V; Sjoberg, D. D.; Pehrke, D.; Steuber, T.; Huland, H.; Graefen, M.; Scardino, P. T.; Schlomm, T.; Vickers, J. A.; Lilj a, H.; Sauter, G. A pre-specified model based on four Kallikrein markers in blood improves predictions of adverse pathology and biochemical recurrence after radical prostatectomy. Br. J. Cancer 2020, 123, 604-609.
(56) McKiernan, J.; Donovan, M. J.; O'Neill, V; Bentink, S.; Noerholm, M.; Belzer, S.; Skog, J.; Kattan, M. W.; Partin, A.; Andriole, G.; Brown, G.; Wei, J. T.; Thompson Jr, I. M.; Carroll, P. A novel urine exosome gene expression assay to predict high-grade prostate cancer at initial biopsy. JAMA Oncol. 2016, 2, 882-889.
(57) Sun, S.; Hu, Y.; Jia, L.; Eshghi, S. T.; Liu, Y.; Shah, P.; Zhang, H. Site-specific profiling of serum glycoproteins using N-linked glycan and glycosite analysis revealing atypical N-glycosylation sites on albumin and α-1B-glycoprotein. Anal Chem. 2018, 90, 6292-6299.
(58) Moremen, K. W.; Ramiah, A.; Stuart, M.; Steel, J.; Meng, L.; Forouhar, F.; Moniz, H. A.; Gahlay, G.; Gao, Z.; Chapla, D. Expression system for structural and functional studies of human glycosylation enzymes. Nat. Chem. Biol. 2018, 14, 156-162.

1. A method for measuring the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample, the method comprising;
a) Extracting PSA from the blood serum or plasma sample using an anti-PSA antibody;
b) Performing enzymatic treatment of a first portion of the extracted PSA with an α2-3-linked Neu5Ac specific neuraminidase that specifically removes α2-3-linked Neu5Ac to obtain a first PSA extract;
c) Performing enzymatic treatment of a second portion of the extracted PSA with a non-specific neuraminidase capable of removing both α2-3- and α2-6-linked Neu5Ac to obtain a second PSA extract;
d) Measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the following:
i) a portion of the extracted PSA that was not enzymatically treated with a neuraminidase; and in
ii) the first and second PSA extracts; and
e) Quantification of the fractional amount of α2-3-linked Neu5Ac in PSA from the CoM analysis.

2. The method of claim 1, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

3. The method of claim 1 further comprising
f) Using the fractional amount of total Neu5Ac on PSA that is α2-3-linked Neu5Ac to predict the subject as having clinically significant prostate cancer (Gleason Group 2, 3, 4 and 5 also known as GG2-5);
i. wherein when the fraction (percentage) of Neu5Ac on PSA that is α2-3-linked Neu5Ac is less than 0.240 (24.0%), then the subject has a high likelihood (>95% chance) of low risk (GG1) prostate cancer or no prostate cancer;
ii. wherein when the fraction (percentage) is equal to or above 0.240 (24.0%) and less than or equal to 0.280 (28.0%), then the subject has a low likelihood of clinically significant prostate cancer (GG2-5); and
iii. wherein when the fraction (percentage) is above 0.280 (28.0%), then the subject has a high likelihood (˜90%) of clinically significant prostate cancer (GG2-5).

4. The method of claim 3, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

5. A method of determining whether a human subject suspected of having prostate cancer should receive a needle biopsy, the method comprising:
a) Extracting PSA from the blood serum or plasma sample using an anti-PSA antibody;
b) Performing enzymatic treatment of a first portion of the extracted PSA with an α2-3-linked Neu5Ac specific neuraminidase that specifically removes α2-3-linked Neu5Ac to obtain a first PSA extract;
c) Performing enzymatic treatment of a second portion of the extracted PSA with a non-specific neuraminidase capable of removing both α2-3- and α2-6-linked Neu5Ac to obtain a second PSA extract;
d) Measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the following:
i) a portion of the extracted PSA that was not enzymatically treated with a neuraminidase; and in
ii) the first and second PSA extracts; and
e) Quantification of the fractional amount of α2-3-linked Neu5Ac in PSA from the CoM analysis; and
i. when the fraction (percentage) of total Neu5Ac on PSA that is α2-3-linked Neu5Ac is greater than 0.280 (28.0%) recommend the subject proceed immediately to a needle biopsy and forego the need for a diagnostic MRI scan; and
ii. when the fraction (percentage) is less than 0.240 (24.0%) recommend no diagnostic MRI scan and no needle biopsy.

6. The method of claim 5, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

7. The method of claim 5 wherein when the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated; and if the fraction (percentage) of fucosylated PSA is greater than 0.640 (64.0%), then recommend the subject have a needle biopsy and recommend to forego an mpMRI scan; and if the fraction (percentage) of fucosylated PSA is less than or equal to 0.640 (64.0%), then recommend the subject have an mpMRI; and when the mpMRI shows a PI-RADS score of 3, 4 or 5, then recommend a needle biopsy and if the PI-RADS score is 1 or 2 then recommend no needle biopsy.

8. The method of claim 5 further comprising providing a treatment
i. when the fraction (percentage) of total Neu5Ac on PSA that is α2-3-linked Neu5Ac is greater than 0.280 (28.0%) treat the subject with a needle biopsy and forego a diagnostic MRI scan; and
ii. when the fraction (percentage) is less than 0.240 (24.0%) do not treat subject to a needle biopsy or diagnostic MRI scan.

9. The method of claim 8, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

10. The method of claim 8 wherein when the fraction (percentage) of total Neu5Ac on PSA that is α2-3-linked Neu5Ac falls between ≥0.240 (24.0%) and ≤0.280 (28.0%), then measure the relative amount of PSA in the subject's blood serum or plasma that is fucosylated; and
when the fraction (percentage) of fucosylated PSA is greater than 0.640 (64.0%), then treat the subject with a needle biopsy and do not treat with an mpMRI scan; and
when the fraction (percentage) of fucosylated PSA is less than or equal to 0.640 (64.0%), then treat the subject with an mpMRI; and
when the mpMRI shows a PI-RADS score of 3, 4 or 5, then treat the subject with a needle biopsy; and
when the PI-RADS score is 1 or 2 do not treat the subject with a needle biopsy.

11. The method of claim 10, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

12. A method of monitoring and treating a prostate cancer patient post prostate cancer removal surgery for need of additional cancer treatment, the method comprising:
a) Extracting PSA from the blood serum or plasma sample using an anti-PSA antibody;
b) Performing enzymatic treatment of a first portion of the extracted PSA with an α2-3-linked Neu5Ac specific neuraminidase that specifically removes α2-3-linked Neu5Ac to obtain a first PSA extract;
c) Performing enzymatic treatment of a second portion of the extracted PSA with a non-specific neuraminidase capable of removing both α2-3- and α2-6-linked Neu5Ac to obtain a second PSA extract;
d) Measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the following:
i) a portion of the extracted PSA that was not enzymatically treated with a neuraminidase; and in
ii) the first and second PSA extracts; and
e) Quantification of the fractional amount of α2-3-linked Neu5Ac in PSA from the CoM analysis; and when the fraction (percentage) of total Neu5Ac on PSA that is α2-3-linked Neu5Ac is greater than 0.280 (28.0%) recommend or treat the prostate cancer patient with additional cancer treatment; and when the total Neu5Ac on PSA that is α2-3-linked Neu5Ac falls between ≥0.240 (24.0%) and ≤0.280 (28.0%), perform intensified monitoring post-treatment; and wherein when the fraction (percentage) of total Neu5Ac on PSA that is α2-3-linked Neu5Ac is less than 0.240 (24.0%), then no perform no monitoring post-treatment.

13. The method of claim 12, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

14. A method for measuring the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample, the method comprising;
a) Extracting PSA from the blood serum or plasma sample using an anti-PSA antibody to obtain a PSA extraction sample;
b) Measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the PSA extraction sample;
c) Performing an enzymatic treatment on the PSA extraction sample with an α2-3-linked Neu5Ac specific neuraminidase that specifically removes α2-3-linked Neu5Ac to obtain a first treated PSA extraction sample and measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in first treated PSA extraction sample;
d) Performing an enzymatic treatment on the first treated PSA extraction sample with a non-specific neuraminidase capable of removing both α2-3- and α2-6-linked Neu5Ac to obtain a second PSA extraction sample and measuring and quantifying the relative amount of the total Neu5Ac that is α2-3-linked Neu5Ac wherein the measuring and quantifying are performed with electrospray ionization mass spectrometry (ESI-MS) and center-of-mass (CoM) analysis of the PSA that is present in the second PSA extraction sample; and
e) Quantification of the fractional amount of α2-3-linked Neu5Ac in PSA from the CoM analyses performed in steps b), c), and d).

15. The method of claim 14, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

16. The method of claim 14 further comprising quantifying the relative amounts of α2-3-linked N-acetylneuraminic acid (α2-3-linked Neu5Ac) and α2-6-linked N-acetylneuraminic acid (α2-6-linked Neu5Ac) on PSA present in a blood serum or plasma sample obtained from a human subject suspected of having prostate cancer to characterize whether the subject has a low risk (GG1), intermediate risk (GG2) or a high risk (GG3, 4 or 5) prostate cancer, the method comprising:
f) Using the fractional amount of total Neu5Ac on PSA that is α2-3-linked Neu5Ac to predict the subject as having clinically significant prostate cancer (Gleason Group 2, 3, 4 and 5 also known as GG2-5);
i. wherein when the fraction (percentage) of Neu5Ac on PSA that is α2-3-linked Neu5Ac is less than 0.240 (24.0%), then the subject has a high likelihood (>95% chance) of low risk (GG1) prostate cancer or no prostate cancer;
ii. wherein when the fraction (percentage) is equal to or above 0.240 (24.0%) and less than or equal to 0.280 (28.0%), then the subject has a low likelihood of clinically significant prostate cancer (GG2-5); and
iii. wherein when the fraction (percentage) is above 0.280 (28.0%), then the subject has a high likelihood (˜90%) of clinically significant prostate cancer (GG2-5).

17. The method of claim 16, wherein the α2-3-linked Neu5Ac specific neuraminidase is NEUS enzyme from Streptococcus pneumonia (NanB subtype) and wherein the non-specific neuraminidase capable of removing both α2-3-linked and α2-6-linked Neu5Ac is NEUC enzyme from Clostridium perfringens (NanI subtype).

18.-28. (canceled)
